(19) United States
(12) Patent Application Publication
(10) Pub. No.: US 2026/0083365 A1
(43) Pub. Date: Mar. 26, 2026

(54) METHOD AND SYSTEM FOR DETECTING A BIOANALYTE IN THE BLOOD

(71) Applicant: Ramot at Tel-Aviv University Ltd., Tel-Aviv (IL)

(72) Inventors: Fernando PATOLSKY, Tel-Aviv (IL); Nimrod HARPAK, Tel-Aviv (IL); Ella BORBERG, Tel-Aviv (IL); Adva RAZ, Tel-Aviv (IL)

(73) Assignee: Ramot at Tel-Aviv University Ltd., Tel-Aviv (IL)

(21) Appl. No.: 19/106,655

(22) PCT Filed: Aug. 25, 2023
(86) PCT No.: PCT/IL2023/050905

§ 371 Date: Feb. 26, 2025

Related U.S. Application Data
(60) Provisional application No. 63/401,178, filed on Aug. 26, 2022.

(51) Int. Cl. A61B 5/1473; A61B 5/00; A61B 5/145

(52) U.S. Cl. CPC A61B 5/14735; A61B 5/0002; A61B 5/14546; A61B 5/685; A61B 2562/0285

(57) ABSTRACT

A microneedle device comprises a device structure, insertable to a living body and being formed with an opened niche at least partially surrounded by walls of micrometric heights above a base of the niche for allowing the niche to be filled with a blood sample upon the insertion. The device also comprises a biosensor configured to sense a bioanalyte in the blood sample and having a sensing element formed on the base. The thickness of the sensing element is less than the micrometric heights of the walls.

### RELATED APPLICATION

This application claims the benefit of priority of U.S. Provisional Patent Application No. 63/401,178 filed on Aug. 26, 2022, the contents of which are incorporated herein by reference in their entirety.

### FIELD AND BACKGROUND OF THE INVENTION

The present invention, in some embodiments thereof, relates to bio-detection and, more particularly, but not exclusively, to a method and system for detecting a bioanalyte in the blood.

Detection of clinical biomarkers is useful as they can provide critical data regarding an individual's medical condition and may assist, by proper early diagnosis, in managing diseases and preventing mortalities. Modern-day medical diagnosis relies on blood tests as the primary indicator for human health, as blood contains tens of thousands of proteins, biomarkers, and other biological species.

WO 2012/137207 describes a method of measuring a metabolic activity of a cell, effected by independently measuring in an extracellular environment of the cell, time-dependent acidification profiles due to secretion of non-volatile soluble metabolic products; non-volatile soluble metabolic products and volatile soluble metabolic products; and volatile soluble metabolic products, and uses of such a method for diagnosing and monitoring disease treatment.

WO2015/059704 describes a system having a chamber in controllable fluid communication with a sensing compartment. The chamber contains a fluid and the sensing compartment comprises a semiconductor nanostructure and a functional moiety covalently attached to the nanostructure. The functional moiety is such that upon contacting a redox reactive agent, the nanostructure exhibits a detectable change in an electrical property.

### SUMMARY OF THE INVENTION

According to an aspect of some embodiments of the present invention there is provided a microneedle device. The microneedle device comprises a device structure, insertable to a living body and being formed with an opened niche at least partially surrounded by walls of micrometric heights above a base of the niche for allowing the niche to be filled with a blood sample upon the insertion. The device also comprises a biosensor configured to sense a bioanalyte in the blood sample and having a sensing element formed on the base. The thickness of the sensing element is less than the micrometric heights of the walls.

According to some embodiments of the invention the device comprises a plurality of biosensors each configured to sense a different bioanalyte in the blood sample.

According to some embodiments of the invention the device structure is formed with a plurality of opened niches, wherein sensing elements of at least two of the biosensors are on bases of different niches.

According to some embodiments of the invention sensing elements of at least two of the biosensors are on a base of the same niche.

According to some embodiments of the invention the device comprises an inlet fluidic port at a section of the structure that remains outside the body after the insertion, and a fluidic channel extending from the inlet port to the niche, for establishing a flow of washing buffer into the niche in situ.

According to some embodiments of the invention the device comprises an electrical communication port in electrical communication with the biosensor for transmitting signal from the biosensor to a location outside the body, while the biosensor is inside the body.

According to an aspect of some embodiments of the present invention there is provided a device for monitoring at least presence of a bioanalyte. This device comprises a substrate having a skin contact surface, and a plurality of microneedle devices outwardly protruding from the skin contact surface, wherein at least one of the microneedle devices comprises the microneedle device as delineated above and optionally and preferably as further detailed below.

According to some embodiments of the invention the bioanalyte comprises a protein.

According to some embodiments of the invention the bioanalyte comprises an miRNA.

According to some embodiments of the invention the bioanalyte comprises a free-DNA.

According to some embodiments of the invention the bioanalyte comprises an exosome.

According to some embodiments of the invention the bioanalyte comprises a metabolite.

As used herein, a “metabolite” is an intermediate or product of metabolism. The term metabolite is generally restricted to small molecules and does not include polymeric compounds such as DNA or proteins greater than 100 amino acids in length. A metabolite may serve as a substrate for an enzyme of a metabolic pathway, an intermediate of such a pathway or the product obtained by the metabolic pathway.

In preferred embodiments, metabolites include but are not limited to sugars, organic acids, amino acids, fatty acids, hormones, vitamins, as well as ionic fragments thereof. In another embodiment, the metabolite is an oligopeptides (less than about 100 amino acids in length). In still another embodiment, the metabolite is not a peptide or a nucleic acid.

In particular, the metabolites are less than about 3000 Daltons in molecular weight, and more particularly from about 50 to about 3000 Daltons.

The metabolite may be a primary metabolite (i.e. essential to the microbe for growth) or a secondary metabolite (one that does not play a role in growth, development or reproduction, and is formed during the end or near the stationary phase of growth.

According to some embodiments of the invention the bioanalyte comprises an antibody.

According to some embodiments of the invention the bioanalyte comprises a receptor.

According to some embodiments of the invention the biosensor comprises a source electrode and a drain electrode formed on the base or the walls, and wherein the sensing element comprises a nanostructure having a sub-micrometric thickness connecting between the electrodes and being modified by an immobilized affinity moiety selected to interact with the bioanalyte to effect a change in an electrical property of the nanostructure.

According to some embodiments of the invention the biosensor is a transistor and wherein the nanostructure is a channel of the transistor.

According to some embodiments of the invention the affinity moiety comprises an immunogenic moiety.

According to some embodiments of the invention the immunogenic moiety comprises an antibody or a fragment thereof.

According to some embodiments of the invention the immunogenic moiety comprises an antigen and wherein the bioanalyte comprises an antibody to the antigen.

According to some embodiments of the invention the affinity moiety comprises a ligand and the bioanalyte comprises a receptor.

According to an aspect of some embodiments of the present invention there is provided a method of detecting a bioanalyte in the blood of a subject. The method comprises contacting a with the blood of the subject in vivo, wherein the microneedle device is the microneedle device as delineated above and optionally and preferably as further detailed below. The method also comprises extracting the device from the body of the subject, and obtaining a signal from the biosensor thereby detecting a bioanalyte in the blood.

According to an aspect of some embodiments of the present invention there is provided a method of detecting a bioanalyte in the blood of a subject. The method comprises contacting a microneedle device with the blood of the subject in vivo, wherein the microneedle device is the microneedle device as delineated above and optionally and preferably as further detailed below. The method also comprises extracting the device from the body of the subject, washing the biosensor; and detecting the bioanalyte based on a detectable signal received from the biosensor within a time-window beginning a predetermined time period after a beginning time of the washing.

According to an aspect of some embodiments of the present invention there is provided a method of detecting a bioanalyte in the blood of a subject. The method comprises contacting a microneedle device with the blood of the subject in vivo, wherein the microneedle device comprises an inlet fluidic port and a fluidic channel as delineated above and optionally and preferably as further detailed below. The method also comprises washing the biosensor via the fluidic channel, and detecting the bioanalyte based on a detectable signal received from the biosensor within a time-window beginning a predetermined time period after a beginning time of the washing.

According to some embodiments of the invention the device comprises an electrical communication port in electrical communication with the biosensor for transmitting signal from the biosensor to a location outside the body, and the method comprises receiving the signal via the electrical communication port while the biosensor is inside the body.

According to some embodiments of the invention the method comprises extracting the device from the body of the subject, following the washing, and receiving the signal after the extraction.

According to some embodiments of the invention the detecting comprises excluding from the signal any portion generated before the time-window.

According to some embodiments of the invention the beginning of the time-window is defined at a time point at which a rate of change of the signal, in absolute value, is below a predetermined threshold.

According to some embodiments of the thickness of the microneedle device is from about 100 μm to about 300 μm. According to some embodiments the heights of each of the walls is from about 1 μm to about 10 μm. According to some embodiments the thickness of the biosensor is from about 50 nm to about 200 nm. According to some embodiments the width of the nanostructure is from about 50 nm to about 200 nm. According to some embodiments the device structure is configured to ensure penetration of said device structure into intradermal blood capillaries networks in the kin of the subject. For example, the device structure is configured to ensure penetration to a depth of from about 0.5 mm to about 1.5 mm below the skin's surface.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D. Fabrication and characterization of the SiNW-FET-based microneedle array sensor. (A) Schematic illustration of the top-down fabrication process. (B) SEM images of the fabricated needles, the needles are 150 μm in width and about 250 μm in thickness (scale bar: 125 μm). The green inset shows the opening of the SU8 layer that forms the device window (scale bar: 25 μm). The blue inset shows a close-up image on one of the two devices inside the device window. The source-drain pads lie on pads fabricated from the device layer for better contact and surface area. The nanowires are a part of the device layer, laying on the buried oxide, and are 125 nm in width and 75 nm high (scale bar: 5 μm). (C) Electrical characterization of a representative device. The source-drain voltage was swept between −0.4-0.15 V and the gate was kept constant at −0.3V (black curve), −0.2 V (red curve), −0.1 V (green curve), 0 V (blue curve) and 0.1 V (light blue curve). Inset illustrates how the measurement was made, mimicking the ex-vivo experiments as close as possible. (D) Transconductance measurements of 5 individual devices on the same microneedle FET. Vsd was kept constant on 0.1V while the gate was swept between (−0.3) V to 0.4V.

FIGS. 2A-H. Surface modification process. (A) Illustration of modification. Top: microneedles dipped in 150-200 μl, bottom: each needle drop-cast using a microspotter. (B) Schematic illustration of the chemical modification process, with XPS results of different stages of the modification. (C) Schematic illustration of GFP binding to its antibody to test the modification process. (D) Fluorescence microscopy images results of GFP binding to needles after chemical immobilization of GFP-antibody. The needle is shown before (top) and after (bottom) soaking for 10 minutes in 60 nM GFP. (E) Fluorescence intensity of different areas marked in (D) of needles before (orange) and after (green) soaking for 10 minutes in 60 nM GFP. (F) Fluorescence microscopy images of Alexa488 chemically immobilized to needles without SU8 window before (top) and after (bottom) insertion to PDMS. (G) Fluorescence intensity of Alexa488 chemically immobilized to needles with SU8 window before (blue) and after (yellow) insertion to PDMS. (H) Fluorescence intensity of Alexa488 chemically immobilized to needles before (black) and after (red) insertion to PDMS. Without (top) and with SU-8 window (bottom).

FIGS. 3A-E. Microneedle array dimensions and blood contact. (A) Optical image of comparison in size between common 27G needle for venous blood extraction and the proposed microneedle array sensors. Two types of fabricated microneedles length are shown—400 μm and 1 mm. Scale bar: 5 mm. (B) Schematic illustration of microneedle insertion to the forearm. 1 mm microneedle should reach the blood capillaries in the dermis while 400 μm needles will not reach as effectively. (C) Optical images showing the microneedle before (left) and after (right) a blood droplet was placed on the microneedle. Orange inset shows blood is clearly able to enter the SU8 window. (D) Optical image showing the microneedle after insertion to the skin. (E) Schematic illustration of a different possible location for protein detection in the blood. The microneedle array can be used in the finger without or with prior pricking.

FIGS. 4A-D. Skin insertion and contact with blood vessels. (A) Images of the insertion process of the microneedle array sensors into the forearm. (B) Images showing several insertion experiments of 1 mm microneedle array (left) and 0.4 mm microneedle array (right) to the forearm. (C) Summarized results of blood drawing from insertion experiments of 1 mm microneedle array (red) and 0.4 mm microneedle array (green) to the forearm. (D) Statistical distribution of blood drawing success percentage from 50 insertion experiments of 1 mm microneedle array (black) and 0.4 mm microneedle array (red) to the forearm.

FIGS. 5A-H. In-vivo results of different measurements using the microneedle array. (A) Normalized electrical measurements of PSA-spiked PBS solutions and in-vivo measurements in finger-pricked blood. Four measurement cycles were taken once dissociation stabilization of the non-spiked PBS solution was achieved. Black, orange, green, pink, and light blue curves are clean PBS, 0.03 ng/ml, 0.22 ng/ml, 2.22 ng/ml, and 22.2 ng/ml PSA-spiked PBS dissociation curves, respectively. The dissociation phase was conducted in 5% EG in 100 μM phosphate buffer solution. Inset: one cycle close-up view. (B) PSA calibration curve of normalized signal change in comparison to non-spiked blood. (C) Stabilization curves of PSA association at 10 pM (red curve) and 100 pM (blue curve) spiked-PBS solutions. The results indicate that the sensor requires approximately 60 seconds to achieve a differentiable signal. (D) PSA calibration curve of normalized reaction in comparison to non-spiked serum. (E) One cycle close-up view of the normalized electrical measurement results of serum (black curve), 21 ng/ml GFP-spiked PBS solution (green curve), 22 ng/ml cTnI-spiked PBS solution (red curve), and 0.022 ng/ml PSA-spiked PBS solution (blue curve). (F) in-vivo intradermal capillary PSA concentrations were measured in four subjects using the microneedle array (green bars) compared to PSA enzyme-linked immunosorbent assay (ELISA) calibration measurements (blue dots). (G) ex-vivo intradermal capillary cTnI calibration curve of normalized reaction of a microneedle array modified with a specific antibody to cTnI. Inset shows a schematic illustration of multiple receptors modifications on a single microneedle chip array. (H) Top: Deviation measurements performed on a single device via multiple entries to (100 pM spiked serum solution). Bottom: Variance measurements were performed between different devices via normalized response against a 100 pM spiked serum.

FIGS. 6A-C. Laboratory scale 3D printed mount. (A) Illustration of the mount that enables the microneedle-based sensor operation. The mount chip is held for the user to prick his finger using the microneedles, followed by capping of the mount and consecutive washing in the appropriate buffer solution. (B) Optical images show the 3D printed mount and the laboratory-scale system.

FIG. 7. Scanning electron microscopy image of a microneedle tilted view, scale bar: 250 μm.

FIGS. 8A-C. XPS characterization of modification steps. (A) Clean silicon wafer. (B) wafer modifies with amino-silane. (C) wafer modifies with IgG.

FIG. 9. Electrochemical impedance spectroscopy of the modification steps. Measurements were performed using a three-electrode system (silicon piece as working electrode, platinum mesh as counter electrode and Ag/AgCl as a reference electrode) submerged in 0.01×PBS solution, under 20 mV amplitude at 0.1V.

FIGS. 10A-B. Fluorescence microscopy images of Alexa488 chemically immobilized to needles with SU8 window before (A) and after (B) insertion to PDMS.

FIGS. 11A-B. Electrical pH measurements using the microneedle array without fluidic devices. (A) One cycle close-up view of the electrical measurement results of measurements in solutions of pH 4.9-7.5. The microneedles were dipped in 2 ml solution of each pH for 5-10 minutes until stabilization. (B) Relative change in signal in comparison to pH 4.9. The colors in the graph represent the same pH values as in (A).

FIGS. 12A-B. Normalized electrical measurements of PSA-spiked serum and specificity measurements. (A) One cycle close-up view of the electrical measurement results of dissociation stabilization of the non-spiked serum (black curve), and serum spiked with 0.024-24 ng/ml PSA (orange to blue curves). (B) One cycle close-up view of the electrical measurement results of serum (black curve), 21 ng/ml GFP-spiked PBS solution (green curve), 22 ng/ml cTnI-spiked PBS solution (red curve), and 0.022 ng/ml PSA-spiked PBS solution (blue curve). The dissociation phase was conducted in 5% EG in 100 μM phosphate buffer solution.

FIG. 13. One cycle close-up view taken once dissociation stabilization is achieved for clinical PSA measurements of four subjects. The dissociation phase was conducted in 5% EG in 100 μM phosphate buffer solution. Clean refers to bovine serum.

FIG. 14. Enzyme-linked immunosorbent assay calibration curve for PSA concentrations.

FIGS. 15A-B. Normalized electrical measurements of Troponin-spiked serum. (A) One cycle close-up view taken once dissociation stabilization is achieved for cTnI-spiked serum. (B) Response curve derived from (A). The dissociation phase was conducted in 5% EG in 100 μM phosphate buffer solution.

FIG. 16. Variation in normalized response between needles, derived from FIG. 5H.

FIGS. 17A and 17B are schematic illustrations of a top view (FIG. 17A) and a cross-sectional side view (FIG. 17B) of a microneedle device, according to some embodiments of the present invention.

FIG. 18 is a schematic illustration of a sensing element according to some embodiments of the present invention.

FIG. 19 is a schematic illustration of an array of sensing elements according to some embodiments of the present invention.

### DESCRIPTION OF SPECIFIC EMBODIMENTS OF THE INVENTION

The present invention, in some embodiments thereof, relates to bio-detection and, more particularly, but not exclusively, to a method and system for detecting a bioanalyte in the blood.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

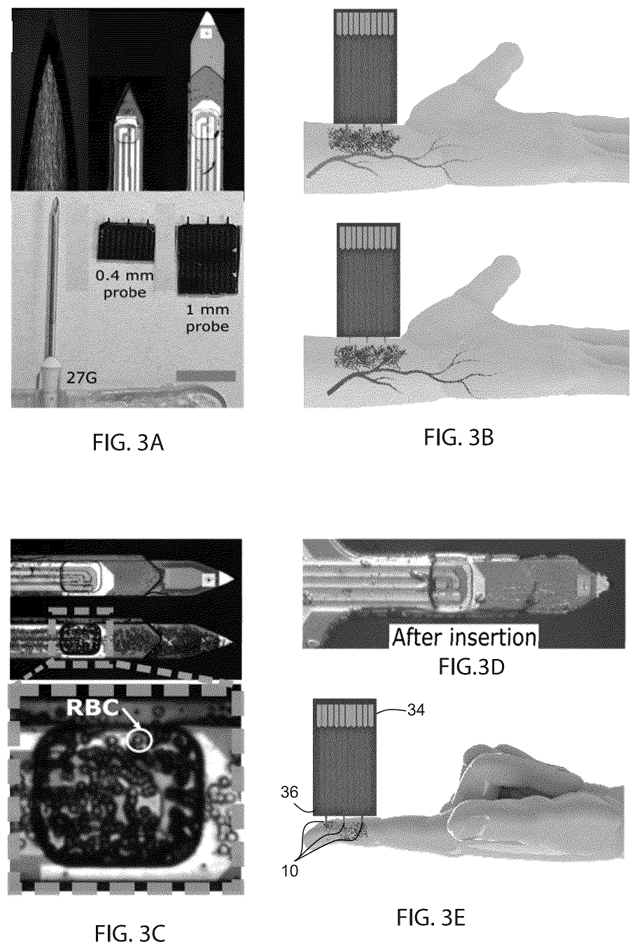

Reference is first made to FIGS. 17A and 17B which are schematic illustrations of a top view (FIG. 17A) and a cross-sectional side view (FIG. 17B) of a microneedle device 10, according to some embodiments of the present invention. The cross-sectional side view of FIG. 17B shows the front part of device 10, in a plane perpendicular to the plane of FIG. 17A and along the line A—A. Microneedle device 10 comprises a device structure 12, insertable to a living body (not shown, see FIGS. 3B, 3E) and being formed with an opened niche 14 at least partially surrounded by walls 16 of micrometric heights h above a base 18 of niche 14. The advantage of having an open niche is that it allows niche 14 to be filled with a blood sample (not shown, see FIG. 3C) upon the insertion of device 10 to the living body. Device 10 also comprises a biosensor 20 configured to sense a bioanalyte in the blood sample. Biosensor 20 can comprise one or more sensing elements 22. As illustrated in FIG. 17B, sensing element 22 formed on base 18 and having a thickness less than the micrometric heights h.

Typical width w for device 10 can be from about 50 μm to about 1 mm, or from about 75 μm to about 800 μm, or from about 75 μm to about 700 μm, or from about 75 μm to about 500 μm, or from about 75 μm to about 400 μm, or from about 75 μm to about 300 μm, or from about 100 μm to about 300 μm, or from about 100 μm to about 200 μm. The typical thickness t of device 10 is about X times larger than the height h of the walls 16, where X is at least 5 or at least 10 or at least 20 or at least 30 or at least 40. For example, t can be from about 100 μm to about 400 μm, and h can be from about 1 μm to about 20 μm. In an embodiment, t is about 250 μm, and h is about 5 μm. Typically, each of the lateral dimensions of niche 14 is independently from about 50 μm to about 1 mm, or from about 75 μm to about 800 μm, or from about 75 μm to about 700 μm, or from about 75 μm to about 500 μm, or from about 75 μm to about 400 μm, or from about 75 μm to about 300 μm, or from about 100 μm to about 300 μm, or from about 100 μm to about 200 μm.

In some embodiments of the present invention device 10 comprises an electrical communication port 34 in electrical communication with biosensor 20 for transmitting signal from biosensor 20 to a location outside the body while biosensor 20 is inside the body. For clarity of presentation, electrical communication between biosensor 20 and port 34 is not specifically illustrate. Communication port 34 can be of any type that allows wired or wireless communication. For example, communication port 34 can be a USB port or the like.

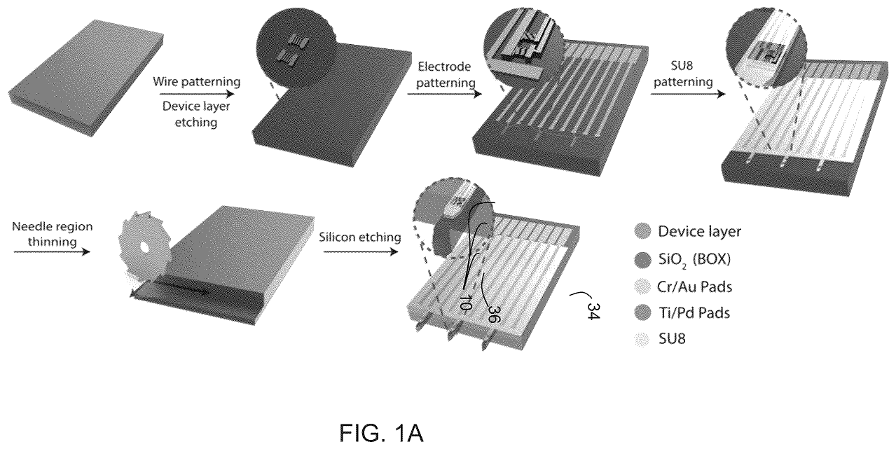

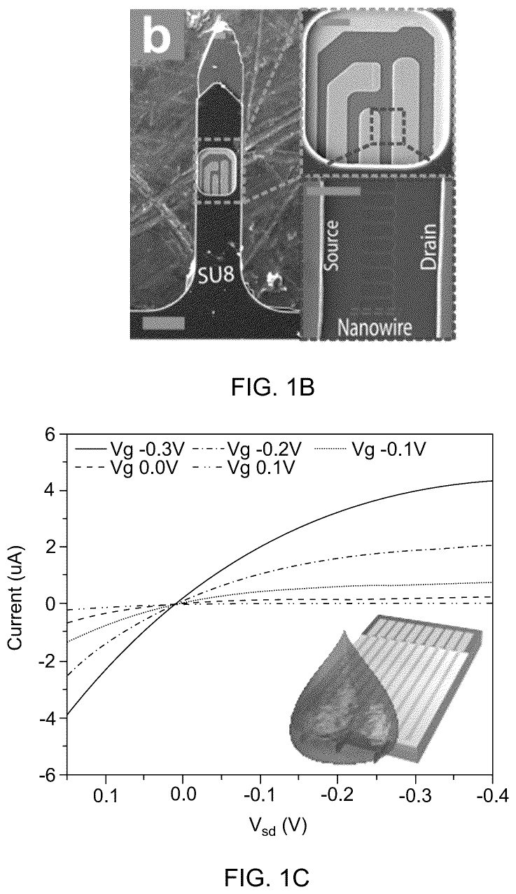

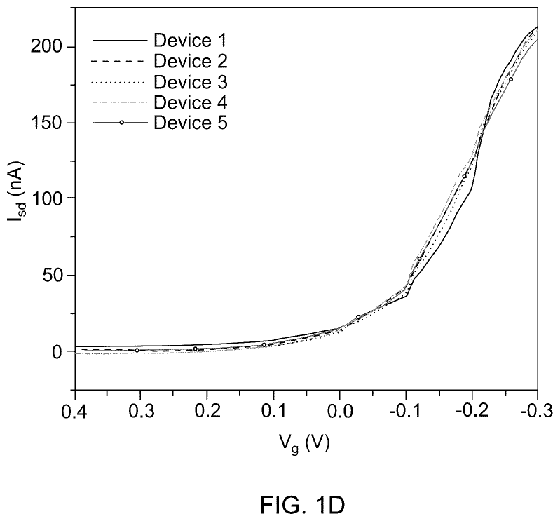

One or more of devices similar to device 10 can be mounted or formed on a surface, such as, but not limited to, a skin contact surface, in a manner that the microneedle devices outwardly protrude from the surface. A representative illustration of these embodiments is shown in FIGS. 1A and 3E showing a plurality of microneedles 10 (three, in this example) protruding out of a surface 36.

The microneedle device 10 can be constructed from any of a variety of materials, including, without limitation metals, ceramics, semiconductors, organics, polymers, and composites. Preferred materials of construction include pharmaceutical grade stainless steel, gold, titanium, nickel, iron, tin, chromium, copper, palladium, platinum, alloys of these or other metals, silicon, silicon dioxide, and polymers. The microneedle preferably has a mechanical strength to remain intact while being inserted into the skin 30, while remaining in place, and while being removed. The microneedle is preferably sterile. Any sterilization procedure can be employed, including, without limitation, ethylene oxide or gamma irradiation.

In case of more than one microneedle (e.g., a plurality of microneedles protruding of the same surface), the microneedles of the plurality may include microneedles having various lengths, base portion materials, body portion diameters (i.e., gauge), tip portion shapes, spacing between microneedles, coatings, etc.

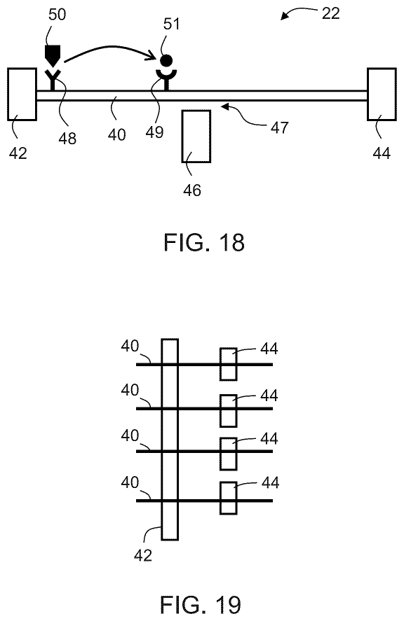

Sensing element 22 preferably comprises an affinity moiety and a nanostructure having a sub-micrometric thickness modified by a functional moiety. A magnified schematic illustration of sensing element 22 according to some embodiments of the present invention is shown in FIG. 18. Shown in FIG. 18 are an affinity moiety 48 and a nanostructure 40 modified by a functional moiety 49. Functional moiety 49 is optionally and preferably covalently attached to nanostructure 40. Affinity moiety 48 is effective to react (e.g., bind) specifically to a bioanalyte 50, to produce to produce a reaction product 51 which in turn reacts with functional moiety 49. In some embodiments, bioanalyte 50 reacts directly with functional moiety 49, in which case it is not necessary for element 22 to comprise affinity moiety 48 on nanostructure 40.

Representative examples of types bioanalyte that can be sensed by element 22 include, without limitation, a protein, an miRNA, a free-DNA, an exosome, a metabolite, an antibody, and a receptor.

Functional moiety 49 is optionally and preferably a moiety that is capable of reacting with reaction product 51 and change, optionally and preferably in a reversible manner, one or more of the electrical property of nanostructure 40 as a result of this reaction. Representative examples of functional moieties suitable for use as functional moiety 49 according to some embodiments of the present invention are found in International Patent Application, Publication No. WO2015/059704, the contents of which are hereby incorporated by reference.

Nanostructure 40 is preferably elongated.

As used herein, a “elongated nanostructure” generally refers to a three-dimensional body which is made of a solid substance, and which, at any point along its length, has at least one cross-sectional dimension and, in some embodiments, two orthogonal cross-sectional dimensions less than 1 micron, or less than 500 nanometers, or less than 200 nanometers, or less than 150 nanometers, or less than 100 nanometers, or even less than 70, less than 50 nanometers, less than 20 nanometers, less than 10 nanometers, or less than 5 nanometers. In some embodiments, the cross-sectional dimension can be less than 2 nanometers or 1 nanometer.

In some embodiments, the nanostructure has at least one cross-sectional dimension ranging from 0.5 nanometers to 200 nanometers, or from 1 nm to 100 nm, or from 1 nm to 50 nm.

The length of a nanostructure expresses its elongation extent generally perpendicularly to its cross-section. According to some embodiments of the present invention the length of the nanostructure ranges from 10 nm to 50 microns.

The cross-section of the elongated nanostructure may have any arbitrary shape, including, but not limited to, circular, square, rectangular, elliptical and tubular. Regular and irregular shapes are included.

In various exemplary embodiments of the invention the nanostructure is a non-hollow structure, referred to herein as “nanowire”.

A “wire” refers to any material having conductivity, namely having an ability to pass charge through itself.

In some embodiments, a nanowire has an average diameter that ranges from 0.5 nanometers to 200 nanometers, or from 1 nm to 100 nm, or from 1 nm to 50 nm.

In some embodiments of the present invention, the nanostructure is shaped as hollow tubes, preferably entirely hollow along their longitudinal axis, referred to herein as “nanotube” or as “nanotubular structure”.

The nanotubes can be single-walled nanotubes, multi-walled nanotubes or a combination thereof.

In some embodiments, an average inner diameter of a nanotube ranges from 0.5 nanometers to 200 nanometers, or from 1 nm to 100 nm, or from 1 nm to 50 nm.

In case of multi-walled nanotubes, in some embodiments, an interwall distance can range from 0.5 nanometers to 200 nanometers, or from 1 nm to 100 nm, or from 1 nm to 50 nm.

It is appreciated that while FIG. 18 shows a single nanostructure 40, some embodiments contemplate a configuration in which biosensor 20 comprises a plurality (i.e., two or more) of nanostructures. When a plurality of nanostructures is employed, the nanostructures 40 are optionally and preferably arranged in an array. For example, the nanostructures can be arranged generally parallel to each other, as schematically illustrated in FIG. 19.

Selection of suitable materials for forming nanostructure 40 as described herein will be apparent and readily reproducible by those of ordinary skill in the art, in view of the guidelines provided herein for beneficially practicing embodiments of the invention. For example, nanostructure 40 of the present embodiments can be made of an elemental semiconductor of Group IV, and various combinations of two or more elements from any of Groups II, III, IV, V and VI of the periodic table of the elements.

As used herein, the term “Group” is given its usual definition as understood by one of ordinary skill in the art. For instance, Group III elements include B, Al, Ga, In and Tl; Group IV elements include C, Si, Ge, Sn and Pb; Group V elements include N, P, As, Sb and Bi; and Group VI elements include O, S, Se, Te and Po.

In some embodiments of the present invention the nanostructure is made of a semiconductor material, optionally and preferably a semiconductor material that is doped with donor atoms, known as “dopant”. The present embodiments contemplate doping to effect both n-type (an excess of electrons than what completes a lattice structure lattice structure) and p-type (a deficit of electrons than what completes a lattice structure) doping. The extra electrons in the n-type material or the holes (deficit of electrons) left in the p-type material serve as negative and positive charge carriers, respectively. Donor atoms suitable as p-type dopants and as n-type dopants are known in the art.

For example, the nanostructure can be made from silicon doped with, e.g., B (typically, but not necessarily Diborane), Ga or Al, to provide a p-type semiconductor nanostructure, or with P (typically, but not necessarily Phosphine), As or Sb or to provide an n-type semiconductor nanostructure.

In some embodiments of the present invention the nanostructure is made of, or comprises, a conductive material, e.g., carbon. For example, the nanostructure can be a carbon nanotube, either single-walled nanotubes (SWNT), which are can be considered as long wrapped graphene sheets, or multi walled nanotubes (MWNT) which can be considered as a collection of concentric SWNTs with different diameters. A typical diameter of a SWNT is less of the order of a few nanometers and a typical diameter of a MWNT is of the order of a few tens to several hundreds of nanometers.

When a plurality of nanostructures is employed, the nanostructures can be grown using, for example, chemical vapor deposition. Alternatively, the nanostructures can be made using laser assisted catalytic growth (LCG). Any method for forming a semiconductor nanostructure and of constructing an array of a plurality of nanostructures is contemplated. When a plurality of nanostructures 40 is employed, there is an affinity moiety 48 immobilized on each of the nanostructures. In some embodiments of the present invention all the affinity moieties are the same across all the nanostructures, and in some embodiments at least two nanostructures are attached to different affinity moieties.

A reaction event between reaction product 51 and moiety 49 (or between bioanalyte 50 and moiety) changes the surface potential of nanostructure 40 and therefore results in a change of an electrical property of nanostructure 40. For example, nanostructure 40 can exhibit a change in density of electrons or holes over some region of nanostructure 40 or over the entire length of nanostructure 40. Nanostructure 40 can additionally or alternatively exhibit a change in its conductivity or resistivity.

The change electrical property of nanostructure 40 can be monitored according to some embodiments of the present invention by an arrangement of electrodes, thereby allowing an indirect monitoring the presence, absence or level of bioanalyte 50 in the blood sample, via the reaction of product 51 or bioanalyte 50 with moiety 49. The electrodes can be formed on base 18 or walls 16. In some embodiments of the present invention sensing element 22 comprises a source electrode 42 and a drain electrode 44, wherein nanostructure 40 is disposed between electrodes 42 and 44 and serves as a charge carrier channel. Optionally, sensing element 22 also comprises a gate electrode 46, forming, together with electrodes 42 and 44 and nanostructure 40, a transistor, e.g., a field effect transistor (FET). The gate electrode 46 is optionally and preferably, but not necessarily, spaced apart from nanostructure 40 by a gap 47. A gate voltage can be applied to channel nanostructure 40 through gate electrode 46. In some embodiments, when the voltage of gate electrode 46 is zero, nanostructure 40 does not contain any free charge carriers and is essentially an insulator. As the gate voltage is increased, the electric field caused attracts electrons (or more generally, charge carriers) from source electrode 42 and drain electrode 44, and nanostructure 40 becomes conducting. In some embodiments, no gate voltage is applied and the change in the charge carrier density is effected solely by virtue of the interaction between affinity moiety 48 and bioanalyte 50.

In some embodiments, affinity moiety 48 and bioanalyte 50 are members of an affinity pair, wherein moiety 48 is capable of reversibly or non-reversibly binding with high affinity (characterized by a Kd (Dissociation constant) of, e.g., less than 10⁻⁷ M, e.g., less than 10⁻⁸ M, less than 10⁻⁹, less than 10⁻¹⁰ M) to bioanalyte 50. For example, the affinity pair can be an enzyme-substrate pair, a polypeptide-polypeptide pair (e.g., a hormone and receptor, a ligand and receptor, an antibody and an antigen, two chains of a multimeric protein), a polypeptide-small molecule pair (e.g., avidin or streptavidin with biotin, enzyme-substrate), a polynucleotide and its cognate polynucleotide such as two polynucleotides forming a double strand (e.g., DNA-DNA, DNA-RNA, RNA-DNA), a polypeptide-polynucleotide pair (e.g., a complex formed of a polypeptide and a DNA or RNA e.g., aptamer), a polypeptide-metal pair (e.g., a protein chelator and a metal ion), a polypeptide and a carbohydrate (leptin-carbohydrate), and the like.

It is appreciated that when the electrical property of the nanostructure varies in response to the binding between the affinity moiety and the bioanalyte, a detectable signal can be produced. For example, a change in the electrical property of the channel induces a change in the characteristic response of the transistor to the gate voltage (e.g., the source-drain current as a function of the time for a fixed gate voltage, or a fixed source-drain voltage), which change can be detected and analyzed.

In some embodiments of the present invention biosensor 20 comprises a plurality of sensing elements 22 each configured to sense a different bioanalyte in the blood sample. For example, each sensing element can include an affinity moiety that reacts specifically with a different type of bioanalyte. The sensing elements 22 can be on the base of the same niche, as illustrated in FIG. 17A. It is appreciated that while FIGS. 17A and 17B show a single niche, this need not necessarily be the case, since, in some embodiments device structure 12 is formed with a plurality of opened niches 14, wherein sensing elements 22 of at least two of the biosensors 20 are on bases of different niches 14.

In some embodiments of the present invention device 10 is configured in a manner that allows it to be washed in situ. For example, with reference to FIG. 17, device 10 can optionally and preferably comprise an inlet fluidic port 30 at a section of structure 12 that remains outside body after insertion, and a fluidic channel 32 extending from inlet port to niche, for establishing a flow of washing buffer into niche in situ.

In use of device 10, the device is contacted with the blood of the subject in vivo. Thereafter, the device can be extracted from the body of the subject, and a signal can be obtained from the biosensor 20 (e.g., by means of port 34) to detect a bioanalyte in the blood of the subject.

In some embodiments of the present invention the biosensor is washed wherein the bioanalyte is detected based on a detectable signal received from biosensor within a time-window beginning a predetermined time period (e.g., at least 10 seconds or at least 20 seconds or at least 30 seconds or at least 45 seconds or at least 60 seconds or at least 75 seconds or at least 90 seconds or at least 105 seconds or at least 120 seconds or at least 135 seconds or at least 150 seconds) after the beginning time of the washing. Preferably, the detection is based on signal received within the time-window, but is not based on signal received from the sensor before the beginning time of the time-window. The duration of the time-window is preferably from about 30 seconds to about 500 seconds. Other predetermined time periods and time-window durations, including predetermined time periods and time-window durations that are outside the above ranges, are also contemplated. In embodiments in which device 10 comprises fluid channel 32, the biosensor can be washed by introducing washing buffer through inlet fluidic port 30 in situ while the niche 14 is still inside the body.

According to some embodiments of the invention the signal is monitored (either while the device is still in the body, when the washing is in situ by channel 32, or after it is extracted, when the washing is outside the body) from the beginning of the washing, more preferably from immediately before or immediately after the initiation of the washing, but the beginning of the time-window during which the signals on which the determination of the presence or level of the marker is based, is not at the beginning of the washing. In these embodiments, the method optionally and preferably determines the beginning of the time-window from the signal itself. This can be done, for example, by monitoring the time-dependence of the signal (e.g., slope, plateau, zeroing of some derivative with respect to the time, value of some derivative with respect to the time, etc.), and identifying the beginning of the time-window based on a change in the time-dependence. For example, the method can identify the beginning of the time-window as a time point at which the signal exhibits a decrement, or a time point at which the signal exits a plateau region.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

Protein biomarkers detection is useful for preventive medicine and early detection of illnesses. Convectional detection relies on clinical tests consisting of painful, invasive extraction of large volumes of venous blood, time-consuming post-extraction sample manipulation procedures, and mostly label-based complex detection approaches.

This Example describes a point-of-care (POC) diagnosis paradigm based on the application of intradermal finger prick-based electronic nanosensors arrays for protein biomarkers direct detection and quantification down to the sub-pM range, without the need for blood extraction and sample manipulation steps. The nanobioelectronic array of the present embodiments performs biomarker sensing by a rapid intradermal prick-based sampling of proteins biomarkers directly from the capillary blood pool accumulating at the site of the microneedle puncture, requiring only two minutes and less than one microliter blood sample for a complete analysis. A 1 mm long microneedle element was selected to allow for pain-free dermal sampling with a 100% success rate of reaching and rupturing dermis capillaries.

Micromachining processes and top-down fabrication techniques allow the nanobioelectronic sensor arrays of the present embodiments to provide accurate and reliable clinical diagnostic results using multiple sensing elements in each microneedle and all-in-one direct and label-free multiplex biomarkers detection. Preliminary successful clinical studies performed on human volunteers demonstrated the ability of the detection platform to accurately detect protein biomarkers as a POC detection. The present embodiments can be used for detecting also other clinically relevant circulating biomarkers, such as miRNAs, free-DNAs, exosomes, and small metabolites.

Detection of clinical biomarkers is useful particularly in the field of medicine, as they can provide critical data regarding an individual's medical condition and may assist, by proper early diagnosis, in managing diseases and preventing mortalities. The Inventors found that modern-day medical diagnosis relies on blood tests as the primary indicator for human health, as blood contains tens of thousands of proteins, biomarkers, and other biological species. The Inventors found that conventional processes for reliable detection and quantification of biomarkers require time-consuming and complex separation methods of the bodily fluid in order to separate blood cells and other interrupting constituents¹⁻⁴. Such sample preparation can lead to reduced sensitivities and a lack of reliability in specific assays, along with the incapability to perform point-of-care (POC) analysis⁵⁻⁷.

The current widespread blood tests rely solely on painful and invasive venous blood extraction of several tens of milliliters in volume. Despite the fact that it is currently the preferred diagnostic approach, some of the extracted biosamples are eventually discarded after centrifugation due to technical factors related to sample handling, transportation, storage conditions, and post-extraction manipulation^{8,9}. As the world shifts its attention towards POC medical devices, which will ultimately result in simpler methods of analysis and diagnosis, the Inventers devised more reliable and accurate methods to detect diagnostic biomarkers.

POC testing is performed at the time and place of patient care, and is different from the historical arrangement in which testing was wholly confined to central medical laboratories, which required sending specimens away from the point of care, then waiting hours or days to reach results, during which time care must continue without the desired information.

The device of the present embodiments can measure multiple bio-analytes simultaneously in the same sample, allowing a rapid, low-cost, and reliable quantification.

Many known sensing strategies attempt to realize successful diagnosis based on capillary blood sample analysis^{10,11}. The Inventors found that there problems arise from the minimal volumes needed to be extracted and subsequently required for effective analysis: uncontrollable detrimental effects experienced by the blood samples upon extraction and post-extraction manipulation steps, normally occurring before reaching the final sensing phase, such as clotting and hemolysis^{12,13}, substantial limitations of post-extraction sample manipulation steps originating from the tiny volume of extracted samples, in the range of few microliters, further preventing the application of centrifugation and additional required procedures, and the final incapability to perform multiplexed biomarkers analysis on these small volume samples. All these factors lead to significant analytical artifacts impeding diagnosis.

The Inventors developed a paradigm that quantitatively sample and analyze multiple clinical biomarkers of interest directly from the patient's capillary blood confined to the intradermal space in-vivo, unrestricted to current diagnostic technologies requirements of blood samples extraction and post-extraction storage, transportation, and manipulation steps.

Microneedle-based systems have been recently suggested for in-vivo intradermal applications. Due to their size, these systems were demonstrated to be minimally-invasive easy-to-use platforms, where no severe tissue damage is observed by their long-term use¹⁴⁻¹⁷. Most of such systems' applications focused on drug delivery¹⁸⁻²⁰, liquid biosamples extraction for ex-situ analysis^{21,22}, and glucose levels monitoring in diabetic individuals^{23,24}. Currently reported microneedle-based sensing platforms are based on complex non-scalable fabrication procedures, often limiting the resulting devices' reliability, accuracy, and real-world applicability²⁵⁻²⁸. Furthermore, these studies focused on the real-time intradermal detection of small molecular species, mostly glucose. Unlike these systems, the device of the present embodiments is also capable of providing direct in-vivo detection of protein biomarkers from the intradermal space.

Nanowires²⁹⁻³³ have been shown to be a versatile substrate for the fabrication of devices in a broad range of applications such as electronics^{34,35}, optics³⁶, biosciences^{37,38}, medical diagnosis²⁴, and energy storage³⁹⁻⁴¹. More specifically, silicon nanowire-based field-effect transistors (SiNW-FET) are recognized as plausible candidates for label-free, ultrasensitive biosensing devices⁴²⁻⁴⁶, allowing biomarkers detection in the deep sub-pM concentration range, thus covering the clinically relevant biofluid concentrations of most biomarkers of interest.

It is recognized that the intrinsic low limit of detection is achievable only under low-ionic strength conditions due to Debye length screening limitations imposed by the high ionic content of the body fluid under analysis, with ionic strengths higher than 150 mM and Debye length of about 1 nm. This prohibits any practical applications of SiNW-FET devices for sensing unprocessed complex biological fluids. In recent years, successful attempts to overcome the Debye screening length limitation were presented, utilizing the ‘delayed-dissociation’ of surface-bound antigen-antibody pairs^{47,48} and additional approaches^{49,50}. These later studies allowed the sensing of bioanalytes in post-extraction processed whole blood samples, e.g., serum and plasma, as well as directly from unprocessed whole plasma samples in the former case, practically without limiting the analytes that can be quantitatively measured and depending on the surface-modified antibody of choice.

The present embodiments implement and combine SiNW-FET devices with a microneedle-based system.

Such a system enjoys the following advantages: (i) capability to perform highly sensitive sensing of biomolecules, down to the sub-pM range, directly from blood (ii) minimally-invasive probing (iii) rapid measurement times and reliable results for a complete POC device (iv) multiplexed detection of various biomolecules on the same device (v) scalable fabrication.

The microneedle embedded nanosensor arrays of the present embodiments are created by conventional 2D fabrication procedures integrated to fabricate a functional intradermal probing platform. The sensing microneedle probe is capable of impaling the outer dermal layer down to a depth dictated by the microneedle length, rupturing capillaries, and forming a blood pool at the puncture site. A few seconds long intradermal probing of the blood pool by the nanosensor array at the tip of the microneedle element, followed by the ex-vivo detection step, leads to the accurate and quantitative relative biomarker of interest.

This Example presents a fully integrated microneedle-embedded SiNW-FET devices array capable of performing POC rapid label-free sensing of multiple protein biomarkers by a minimally invasive, pain-free method directly from the intradermal space without the requirement for blood extraction and manipulation steps. The fabrication workflow allows for devices redundancy and multiplexed detection, providing reliable results and multi-biomarker detection capabilities by the same sensing platform. This Example demonstrates that by using multifunctional sensing microneedle elements, protein biomarkers detection can be successfully performed directly from the intradermal tiny sub-microliter capillary blood pools filling the impalement sites resulting from the dermal penetration of the microneedle elements. This Example demonstrates diagnosis paradigm, based on the application of microneedle-embedded nanosensors arrays for the blood extraction-free direct intradermal capillary detection of protein biomarkers with a sub-pM sensitivity for all tested species (i.e. below 0.03 ng/ml). This diagnostic platform of the present embodiments can replace the current painful and invasive diagnostic approaches based on blood extraction and manipulation procedures, dominating today's medical blood tests, thus providing a simple POC device for the intradermal capillary rapid and accurate detection of protein biomarkers of interest.

### Results

Silicon-on-insulator (SOI) based devices have been on the rise in the last decade as an alternative to the common bottom-up vapor-liquid-solid (VLS) approach⁵¹⁻⁵³. SOI-based devices exhibit greater reproducibility, lower variability between devices, and can be fabricated using large-scale integration techniques, which enable complex designs to be executed very simply⁵⁴. The robust fabrication process of the microneedle-embedded SiNW-FET device according to some embodiments of the present invention is depicted in FIG. 1A.

An ultrathin device layer of 75 nm silicon-on-insulator (SOI) was selected, with a buried oxide (BOX) thickness of 400 nm. The initial thickness of the dies was 750 μm, in order to maintain the structural integrity of the whole microneedle-embedded device. Once the nanowires were patterned and formed, and the electrodes were fabricated using standard UV lithography and metal evaporation steps, a SU-8 chemically-protecting layer was formed. It should be noted that once the nanowires are formed, no plasma processes were conducted in order to prevent severe ion damage that substantially lower the conductivity⁵⁵.

The SU-8 layer was patterned to leave open access to the sensing devices in the form of a 150 μm×130 μm pool structure. Beyond the potential contamination faced by the nanowires-based devices when impaling the skin, scrubbing of the sensing elements by the intradermal layers may remove the covalently attached molecular biorecognition layer upon impaling into the skin. The heightening of the surface from the nanowires-based devices, by the SU-8 layer, was performed to reduce the likelihood of such a removal. Once the SU-8 layer was formed, mechanical thinning of the needle region was conducted. The nanowire elements based on the SOI device layer may be prone to ion damage, which may ultimately result in loss of conductivity, and the mechanical thinning allowed reducing the time required to etch the final structure into the microneedle elements using deep reactive ion etching (DRIE).

The resulting microneedle-embedded sensors can be seen in the SEM image provided in FIG. 1B. The final needle structure possesses a sharp tip, allowing simple impalement of the skin layers. The final structure is ca. 150 μm wide and 250 μm thick. The SU-8 layer formed is approximately 5 μm thick, not impairing the ability of the needle to penetrate the skin. For the purpose of this example, two sensing elements are fabricated on each needle as can be seen in the insets. In this context, it should be noted that the amount of sensing elements for each needle is limited only by the physical size of the desired needle, therefore numerous devices can be directly fabricated, allowing higher sensing redundancy. The robust fabrication scheme is carefully designed to provide various desired properties in a single on-chip POC device: (i) independent sensing capabilities for each needle, allowing multiplexed detection of various analytes through different chemical modifications, (ii) multiple sensors in each individual needle for redundancy purposes, allowing reliable and accurate diagnostic results and (iii) safekeeping of the sensing region during skin impalement. The ability to fabricate the whole device using common micromachining and lithography tools allows the number of needles, devices, length and shape to be easily varied.

The resulting microneedle-embedded SiNW FET devices were electrically characterized using a probe station. Electrical I-V measurements are shown in FIG. 1C. Using a top-gate characterization method, as illustrated in the inset, the device's electrical characteristics exhibit a p-type behavior, undamaged by the DRIE process used to create the final microneedle structure. Transconductance measurements, depicted in FIG. 1D, show a minor 8.5% variability between different devices.

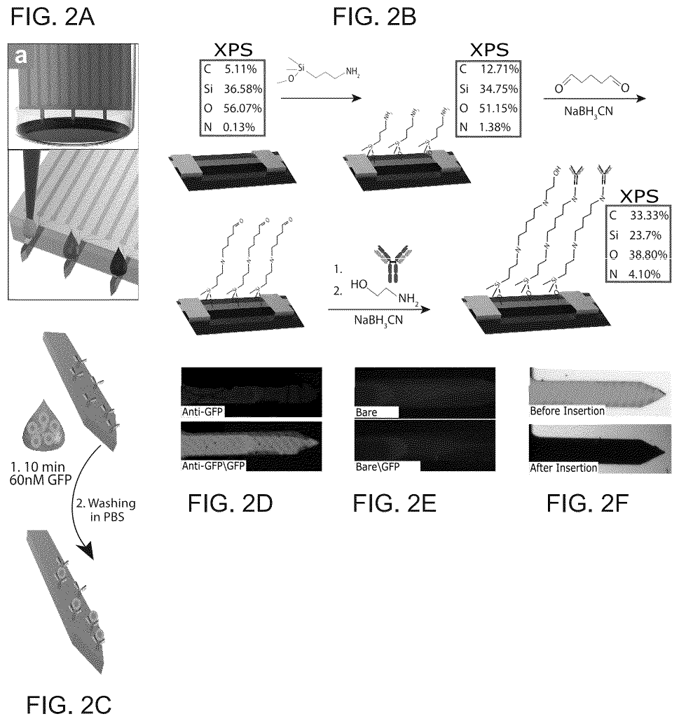

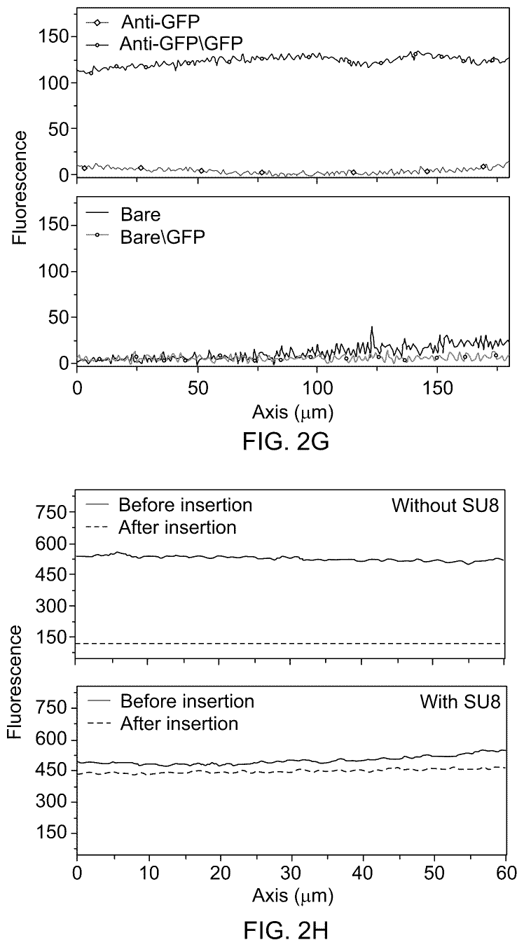

Monitoring different proteins biomarkers is useful for the early detection of many diseases and medical conditions. What usually requires an invasive, painful process of drawing a few milliliters of venous blood for diagnosis can be practically avoided using minimal amounts of capillary blood via rapid antibody-antigen binding followed by electrical measuring. The the microneedle sensing surface was modified with an anti-PSA antibody as a proof-of-concept. Elevated prostate-specific antigen level (PSA) is known to be a biomarker for prostate cancer, considered healthy up to 4 ng/ml (about 120 pM)^{56,57}. Therefore, direct detection of blood-PSA can provide a significant measure for an individual's health without having to extract blood in an invasive and painful manner. As illustrated in FIG. 2A, the chemical modification can be conducted in two ways—either submerging the needles in 150-200 μl antibody solution or by using a micro-spotting system to dispense small volumes of antibody modification solution on each needle individually, allowing for easy multiplexing of the device, as illustrated at the bottom of FIG. 2A.

The modification process is schematically illustrated in FIG. 2B. First, 3-aminopropyldimethylethoxysilane (APDMES) was applied in an inert environment by dipping the needles in the pure silane derivative solution for 1 hour, followed by a thorough wash in toluene and IPA, and placed on a heating plate at 70° C. for 30 minutes in order to evaporate the solvents fully. Then, the needles were dipped in a 200 μL of 10% glutaraldehyde solution in phosphate buffer containing 50 mg of cyanoborohydride for 1 hour and washed thoroughly in IPA and DIW. The needles were then modified with a 30 g/ml anti-PSA solution in phosphate buffer with 50 mg cyanoborohydride at 4° C. The blocking of unreacted aldehyde surface groups was performed via dipping the needles for 2 hours in 200 μL of ethanolamine solution (150 μL in 20 ml phosphate buffer containing 50 mg of cyanoborohydride).

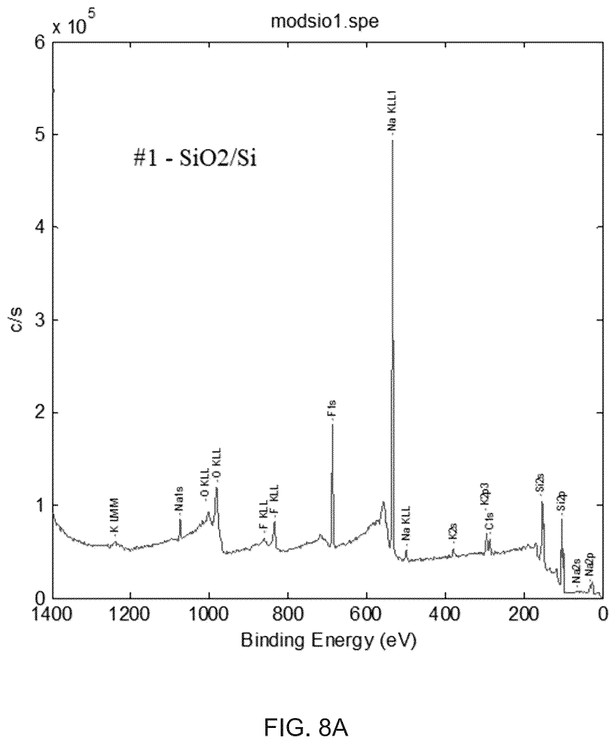

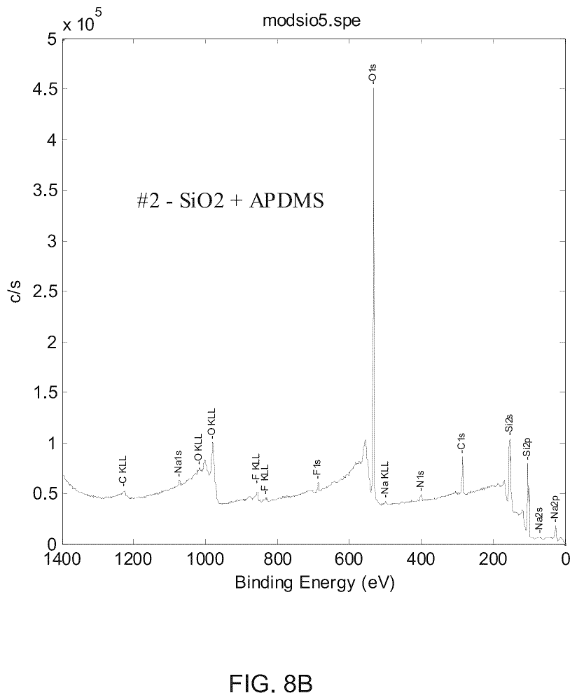

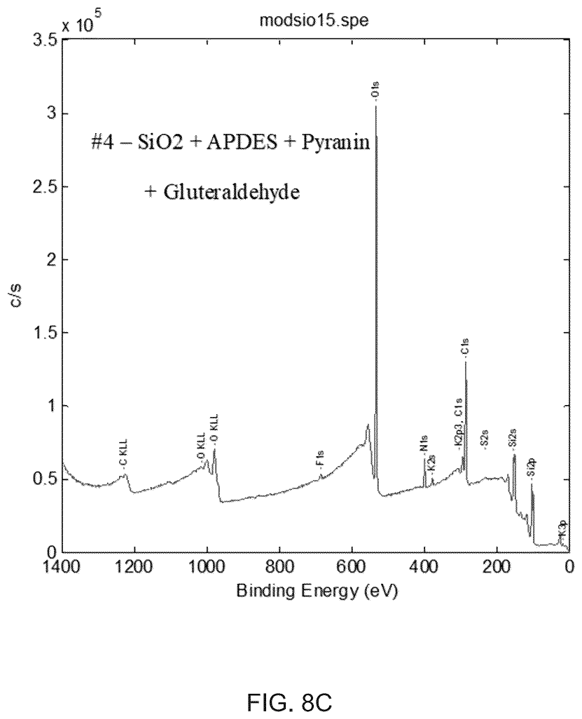

X-ray photoelectron spectroscopy (XPS) analysis results of the different modification steps are shown in FIG. 2B (marked boxes). Once the amino-silane derivative APDMES is covalently attached, a rise in carbon and nitrogen atomic content is measured. A significant increase in carbon and nitrogen content occurs once the glutaraldehyde and antibody molecules are attached to the sensing surface. These XPS results demonstrate the successful modification of the sensing device surface with the receptor antibody molecules. Full XPS spectra are shown in FIGS. 8A-C.

In order to further verify the chemical modification, fluorescence microscopy experiments were conducted. Instead of an anti-PSA antibody, anti-GFP (Green Fluorescent Protein) was modified onto the surface of a bare microneedle. The SU-8 protection layer was not used in this case since the high autofluorescence effect of SU-8 prevents proper fluorescence measurements. The microneedles were then dipped in a 60 nM GFP solution for 10 minutes, as illustrated in FIG. 2C, and further washed in phosphate buffer saline (PBS). As shown in FIG. 2D, 10 minutes of incubation lead to an apparent increase in the fluorescence intensity measured as a result of the specific binding of GFP to the surface of the antibody-modified microneedle element. Six different areas were tested along the needle length axis, marked with dots on FIG. 2D, and fluorescence intensity values were normalized using the average intensity value measured on the chemically unreacted microneedle (not exposed to GFP). The results are plotted in FIG. 2E, showing a about 600% increase in fluorescence intensity. Therefore, it is concluded that the microneedle sensing surface was successfully modified with the antibody receptor molecules.

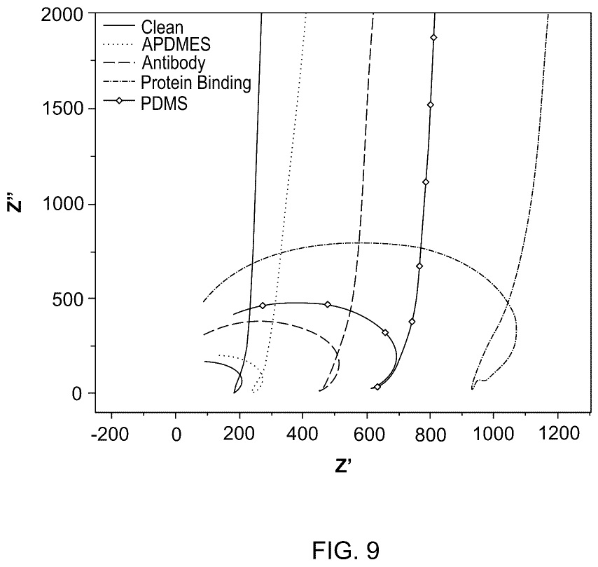

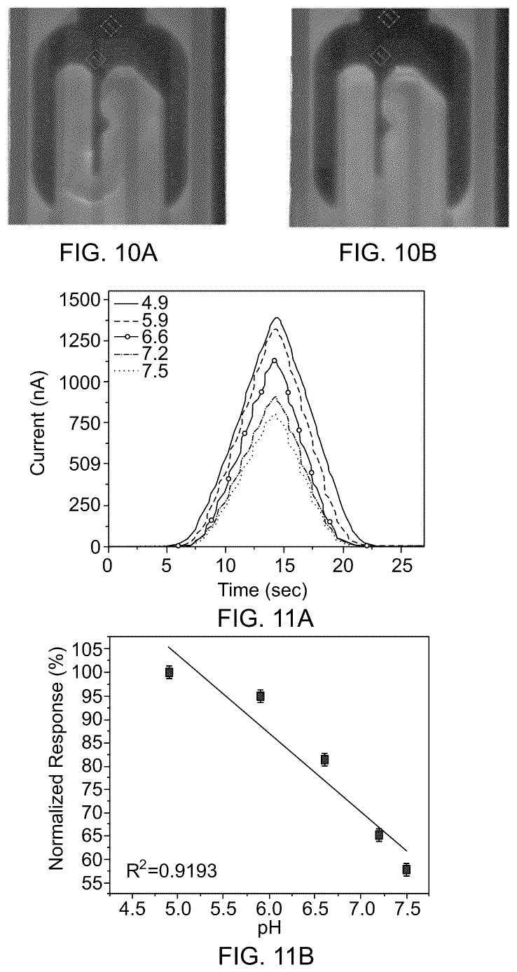

To further verify the modification process, electrochemical impedance spectroscopy (EIS) measurements were performed. The results are shown in FIG. 9. The measurements were performed using a three-electrode system (silicon piece as working electrode, platinum mesh as counter electrode and Ag/AgCl as a reference electrode) submerged in 0.01×PBS solution, under 20 mV amplitude at 0.1V (this potential was chosen as no redox occurs in this point). An increase in the charge transfer resistance was observed as the modification process continued. Once incubated in protein for 10 minutes after antibody modification, a major increase in the charge transfer was observed⁵⁸.

The role of the SU-8 protective layer integrated according to some embodiments of the present invention into the core design of the microneedle-embedded sensors is shown in FIG. 2F. A needle chemically modified with the fluorophore Alexa-488 is shown before (top) and after (bottom) its mechanical insertion into a polydimethylsiloxane (PDMS) skin-mimicking slab. The absence of a SU-8 protection layer leads to complete removal of the antibody biorecognition layer upon insertion of the microneedle elements into this slab, making difficult the successful application of the chemically-modified microneedle devices in the intradermal detection of biomarkers. No measurable difference was measured when performing the same experiment with chemically-modified microneedle elements containing the SU-8 protective layer, as shown in FIG. 2G. These results were also be verified using EIS spectroscopy as shown in FIG. 9. After insertion of the silicon to PDMS (after incubation in protein), a decrease in the charge transfer resistance was observed. These results highlight the advantage of the SU-8 chemistry-protective layer, which allows for microneedle transdermal penetration without compromising neither the electronic devices' performance nor their chemical modification integrity.

Microneedles in the length of 300-500 μm were previously shown to enable in-vivo transdermal monitoring of glucose levels in interstitial fluid (ISF). The Inventors realized that this insertion depth range does not allow microneedle elements to reach and rupture intradermal blood capillaries networks²⁴. Therefore, longer microneedles were fabricated to fully penetrate and rupture the dermal layers and reach capillary depth for the subsequent capillary blood protein biomarkers detection. FIG. 3A shows a comparison of different needles—a 27G needle used in common venous blood extraction procedures, and representative 400 μm-long and 1 mm-long microneedle-embedded SiNW-FET arrays. In today's phlebotomy practices, a needle is inserted up to a few millimeters for the purpose of drawing blood from a vein in the arm. Successful execution of protein biomarkers sensing by the microneedle-based system of the present embodiments can reduce the need for such invasive procedures and can replace these invasive diagnosis means by a minimally invasive and unpainful procedure. FIG. 3B shows a schematic illustration of the difference between 400 μm and 1 mm microneedle. The subcutaneous capillary layer in the forearm and fingers tip is found to be approximately 0.6-1.5 mm in depth⁵⁹, while the epidermis layer is a few hundred micrometers thick⁶⁰. Therefore, a 1 mm needle is expected to penetrate the dermal layer and rupture blood capillaries smoothly in a non-painful way²⁴. Diagnostically, capillary blood protein biomarkers levels have been shown to correlate well with those of venous blood⁶², meaning that the minimally invasive, pain-free procedure by the system of the present embodiments can be used as an alternative to the currently widespread venous blood extraction-based diagnostics approaches.

FIG. 3C shows optical microscope images of a microneedle element before and after its contact with a blood droplet. As shown in the inset, the blood droplet, as evidenced by the presence of red blood cells, rapidly fills the protective window formed by SU-8. Thus, it demonstrates that the SU-8 protective window of the present embodiments does not prevent the blood-to-device free interaction, further allowing the analyte biomarkers detection and quantification. FIG. 3D shows an optical image of a 1 mm microneedle element after skin impalement into the forearm of a volunteer, exhibiting little visual residues along the entire length of the microneedle, indicating full penetration of the needle and reaching the required capillary depth.

Another possible location for in-vivo analysis of protein biomarkers directly from capillary blood is illustrated in FIG. 3E. In recent years, POC diagnostic devices research and development have tried to boost the use of capillary blood instead of venous blood since these tests would be more comfortable on patients and would provide a significant boost in quality-of-life and simplicity of the measurements. Finger pricking has been the method of choice for non-continuous glucose measurements for diabetic individuals. While some research has been conducted on protein biomarkers detection from extracted whole capillary blood samples, these tests require manipulating the small volumes of extracted capillary blood and taking tens of minutes to hours to achieve results^{63,64}. PSA and many other protein biomarkers concentrations in capillary blood correlate well with their respective concentration in venous blood. Therefore, finger pricking can be used for direct in-vivo diagnosis measurements when combined with methods for protein detection in non-manipulated whole capillary blood specimens. Healthy individuals possess a capillary density higher than 60-200 capillaries per mm^{2 65-69}. Thus, the transdermal penetration of the system of the present embodiments, down to the required capillary depth, leads to the rupture of capillary elements and the formation of a tiny capillary blood pool in the site of microneedle puncture. This formed capillary blood pool surrounding the sensing microneedle elements can lead to the surface capture of the protein biomarkers of interest to the electrical nanostructures and their subsequent quantitative detection.

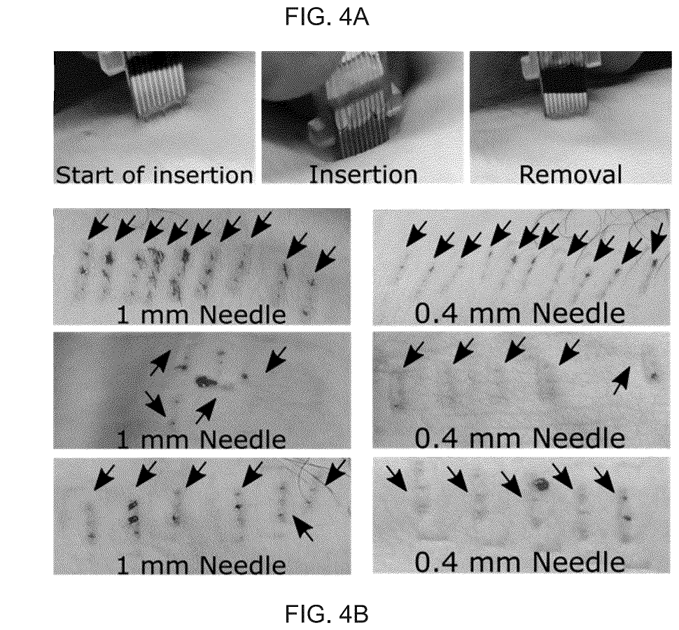

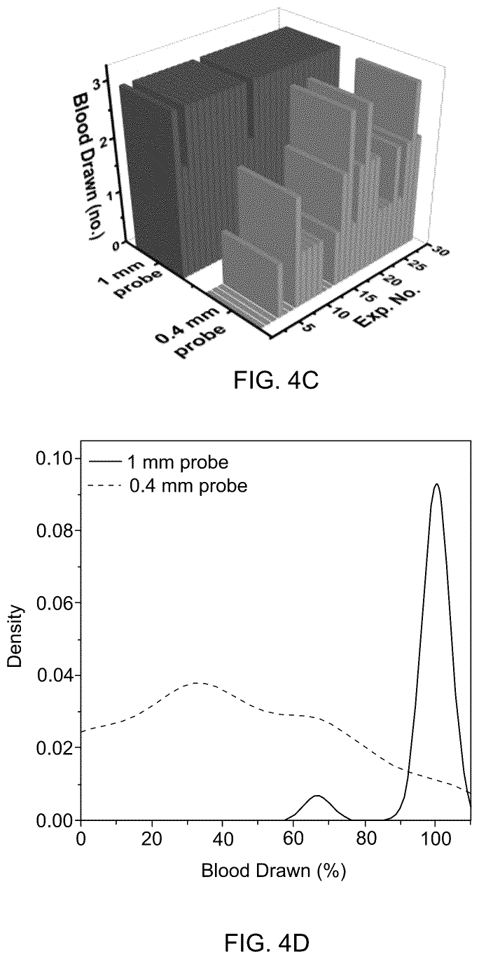

Representative optical images of the microneedle insertion process through the skin in the forearm are shown in FIG. 4A. The needles are shown to penetrate smoothly, with the entire microneedle array inserted in its total length. The chip's handle is used as a stopper component for the ensuring that only the microneedle elements are inserted into the dermal layer. Mechanical tests for quantifying the force needed for skin penetration were previously conducted using a pigs' skin model²⁴, where different skin stiffness was mimicked by using a PDMS support for higher stiffness. Without the PDMS support, 1N force was required to penetrate the skin, and 0.2N was needed with higher stiffness. These results comply with previous microneedle skin penetration experiments.^{24,70,71}. The needles of the present embodiments did not break even after a 5N of applied force, thus proving mechanical robustness and safety.

FIG. 4B displays results from three different healthy volunteers after applying 1 mm (left) and 400 μm (right) microneedle elements to penetrate their skin, while the resulting puncture sites were photographed post-extraction. During insertion, minimal to no pain was reported by all volunteers. Each penetration attempt using 1 mm microneedle elements resulted in the formation of a small drop of capillary blood for all three healthy volunteers tested, indicating that the 1 mm microneedle is sufficient for efficient protein biomarkers measuring purposes directly from capillary blood. Statistics of intradermal capillary blood pool formation experiments comparing the two needles lengths are shown in FIGS. 4C and 4D. A considerably higher success rate for the formation of capillary blood intradermal ‘pools’ is achieved using the longer 1 mm microneedle chips, with almost 100% of all incidents leading to capillary blood pool formation.

Known SiNW-FET sensing devices rely on the use of microfluidics for fluid exchange and sensing assays. In distinction from these devices, no reliance on external microfluidics is required in the microneedle device of the present embodiments. FIG. 11A depicts a representative bare nanodevice's response to different pH media. The other pH solutions were made from 10 mM potassium phosphate monobasic and dibasic species to ensure identical ionic strength, thus eliminating the effects of different ionic strengths on the sensor's signal. Each pH was measured twice by putting the microneedle sensor in the pH solution using a micromanipulator, removing the device, and reinserting it in order to see if any changes occurred. Once the device had reached the first plateau and stabilized, no further difference was seen between each insertion. All the SiNW elements in this Example were coated with a two nm-thick alumina layer by atomic layer deposition. The coating allowed long and stable experiments to be conducted⁷², but devices devoid of such coating are also contemplated. The bare nanodevices exhibit a significant dependence on pH, as shown in FIG. 11B. Additionally, the response variability of the nanodevices is negligible once the signal has stabilized, reaching up to 1% of the total averaged value (average over five cycles measured at the highest point).

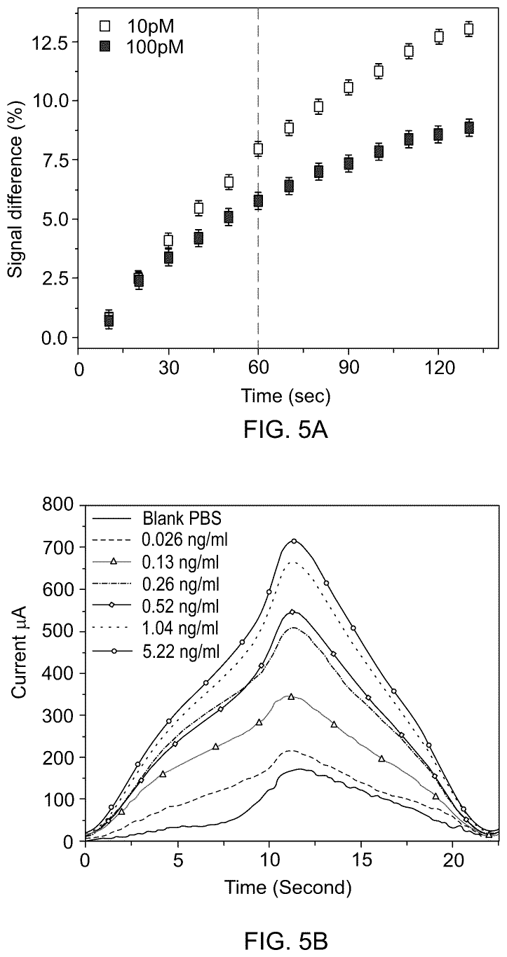

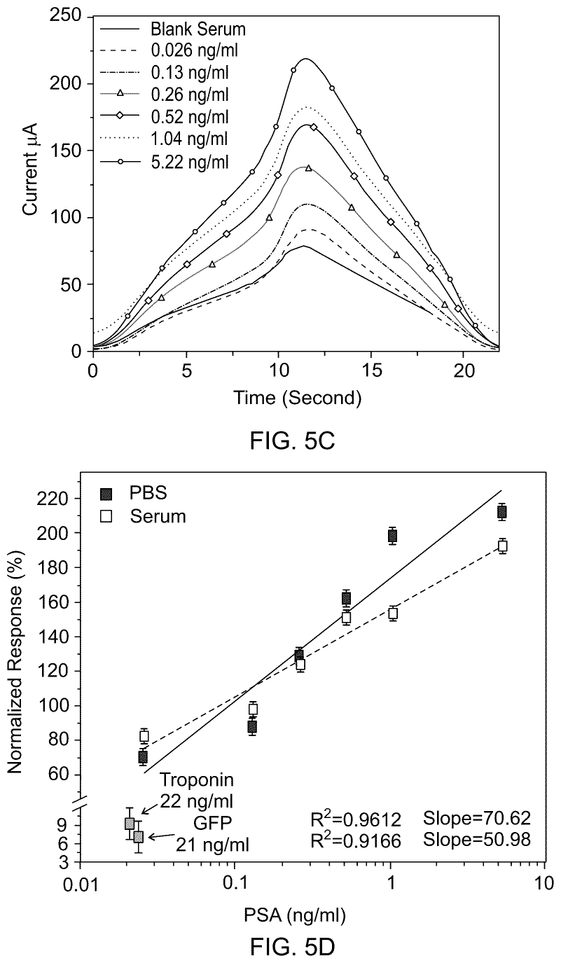

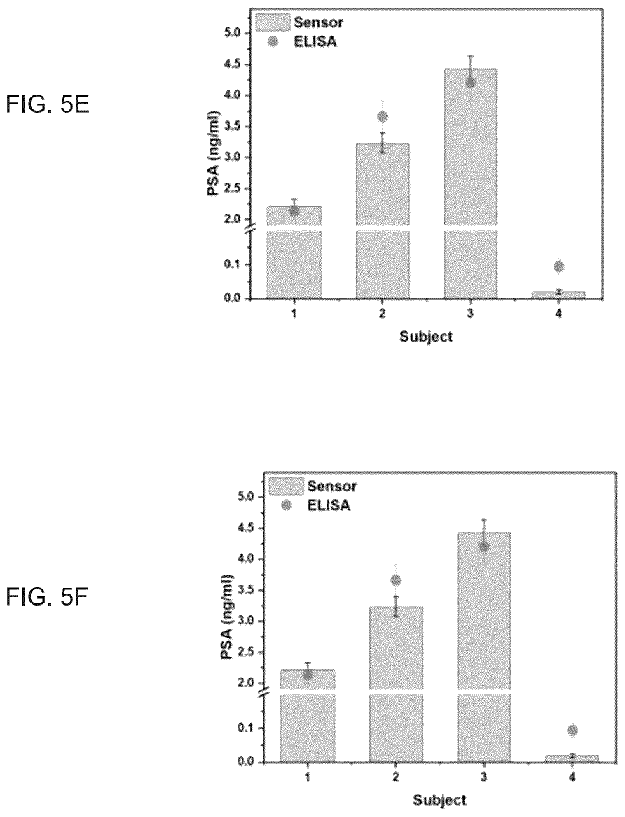

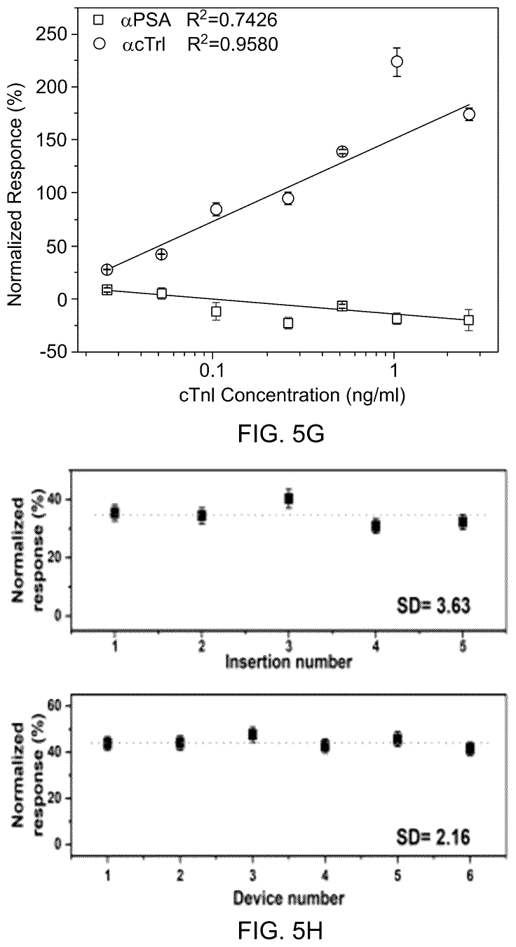

Prior to in-vivo intradermal measurements in capillary blood, an in vitro test was conducted to see the device's sensitivity towards the protein biomarker PSA. The test was separated into two regimes—specific association and dissociation. The association occurred at 1× phosphate buffer saline (PBS) solution spiked with different PSA concentrations. At the same time, the dissociation step was performed using a low ionic strength sensing buffer SB solution (0.01× of 10 mM phosphate buffer) with 5% added ethylene glycol (EG). Prior results using this method have shown the advantage of using EG as a dissociation inhibitor at a concentration of 50%⁴⁷. FIG. 5A shows the result of the in vitro experiments, where the PSA dissociation step was performed using a 1:1 SB:EG solution. The results demonstrate the fast plateau reached after several seconds of PSA association, where an apparent signal differentiation was achieved within 60 seconds. Therefore, the halted transdermal insertion of the microneedle array for only several tens of seconds would be sufficient for diagnostic purposes. Also, a 1:1 SB:EG ratio was chosen to be suitable for the dissociation period, giving slower dissociation rates of the specific biomarker molecules from the surface-confined antibody receptor units, all the while letting non-specific bound moieties dissociate quicker from the sensing area. Therefore, the overall sensing measurement time is fast, with results that can be achieved in less than three minutes, significantly surpassing the time required for today's biomarkers measurements via conventional blood tests.

FIG. 5B depicts one cycle of an in-vitro measurement as described above. The signal normalization was performed against the ‘clean’ signal, measured in a 1×PBS solution without any spiked PSA. FIG. 5C represents one current cycle of in-vitro measurements serum spiked with rising PSA concentrations. Signal normalization was performed against clean unspiked serum. The presented concentration measurements were normalized post-stabilization in relation to the association regime plateau using the formation formula:

$$[ ( {I}_{p} - {I}_{c} ) / {I}_{c} ] * 100 \% \qquad ( EQ . 1 )$$

Where Iₚ is the current received from PSA-containing PBS solutions, and I_{c} is the current received from clean PSA-free PBS solution. Once calculated, the dissociation regimes were normalized using the resulting signal response percentages. The association regime plateau was used for the calibration since dealing with high ionic strength screens the electrical signals originating from protein association due to the short Debye screening length at these conditions. The results corresponding to each concentration were taken in relation to the stabilization of the clean solution's signal.

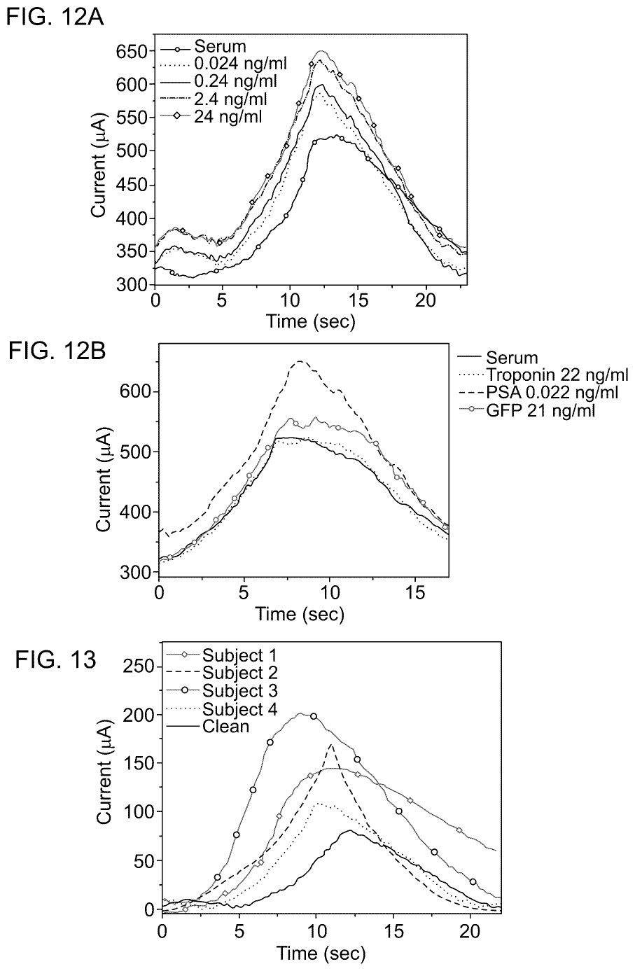

FIG. 5D shows the linear curves of normalized response to different PSA concentrations in spiked PBS buffer (black curve) and in spiked serum (blue curve). As shown, there is a clear concentration-dependent sensing behavior at increasing PSA concentrations. The normalized response is taken as an average value measured after five consecutive cycles of dissociation regime. Specificity tests of the response of the microneedle-embedded SiNW array were conducted by measurements in the presence of high concentrations of non-specific proteins. The anti-PSA modified array showed near-zero response to 22 ng/ml of cardiac troponin I (cTnI) and 21 ng/ml green fluorescent protein (GFP). FIG. 12B compares one cycle of PSA measurement in the presence of 100-fold higher concentrations of GFP and cTnI. These results indicate the high specificity of the sensing microneedle devices of the present embodiments for the specific detection of the desired biomarker antigens. These results indicate low sensitivity to varying interferents, since measurements were performed in untreated serum.

Following in vitro calibration of the sensing devices, in-vivo intradermal sensing measurements through finger pricking, through the 30 seconds-delayed insertion of the microneedle elements into the intradermal space (microneedle impalement is followed by a waiting period of 30 seconds before final removal followed by ex-vivo dissociation measurement) of human volunteers were performed, shown in FIG. 5E (green columns); 4 subjects were tested: Subject 1 is a healthy 30-year-old male, Subject 2 is a healthy 50-year-old male, Subject 3 is a healthy 30-year-old male triathlete (cycling over 20 km weekly), and Subject 4 is a healthy 30-year-old female. The results show that subjects' PSA levels are within the normal healthy range for male Subjects 1 and 2 and are slightly higher for male Subject 3, as was expected since long-distance daily bicycling routines are well known to cause high PSA levels^{73,74}. Very low PSA levels were also expected for the female Subject 4 since normal PSA levels in healthy females are about 0-0.52 ng/ml⁷⁵. The PSA levels from in-vivo microneedle sensing trials were further validated by performing a gold-standard PSA-specific enzyme-linked immunosorbent assay (ELISA) using venous blood from the same volunteers, FIG. 5E (blue dots). ELISA results show that the in-vivo capillary blood direct sensing performed by the microneedle arrays of the present embodiments has the capability to measure the target PSA protein biomarker accurately and falls within the error range of the gold standard ELISA measurements. Additionally, the co-modification of both total-PSA and free-PSA antibody receptors on different microneedle elements on a single sensing chip further could allow the ratiometric clinical assessment of the PSA^{total}/PSA^{free} ratio. This ratio is clinically well known to provide more sensitive and specific information for the diagnosis of prostate cancer.

The microneedle-embedded nanostructure array of the present embodiments poses multiple advantages for POC medical diagnosis applications. Firstly, the lack of need to invasively extract and manipulate blood samples by the direct intradermal capillary blood-based detection of proteins biomarkers provides a great leap in the field of medical diagnosis. This type of sensing platform can also be used in clinical situations where the amount of available blood is inherently small, such as in newborn infants, without the need to prick heels or fingers to extract blood samples for further ex-vivo analysis. Secondly, the simple fabrication process allows redundancy in the number of active sensors, providing reliable results and a small margin of clinical errors.

A large number of functional microneedle elements also allows the multiplexed detection of various protein biomarkers in a single-prick. Each needle can be readily modified with a different antibody or bioreceptor, as illustrated in FIG. 2A. Depending on the multiplexing level required, many antibodies can be modified upon a single microneedle element. This allows multiple biomarkers to be detected using a single-prick single-sensor microneedles-based platform. Results from a multiplexed experiment are shown in FIGS. 5F and 5G, where one needle out of a microneedle array has been modified with a PSA-specific antibody (αPSA) and the adjacent needle was modified with a cTnI-specific antibody (αcTnI). Both needles were exposed to varying concentrations of PSA and cTnI. The needle modified with αPSA showed a clear linear response to PSA, while the needle modified with αcTnI showed no meaningful response, FIG. 5F, and when exposed to cTnI concentrations both the opposite response was exhibited by both needles, FIG. 5G. An additional response curve to cTnI in blood is shown in FIG. 15.

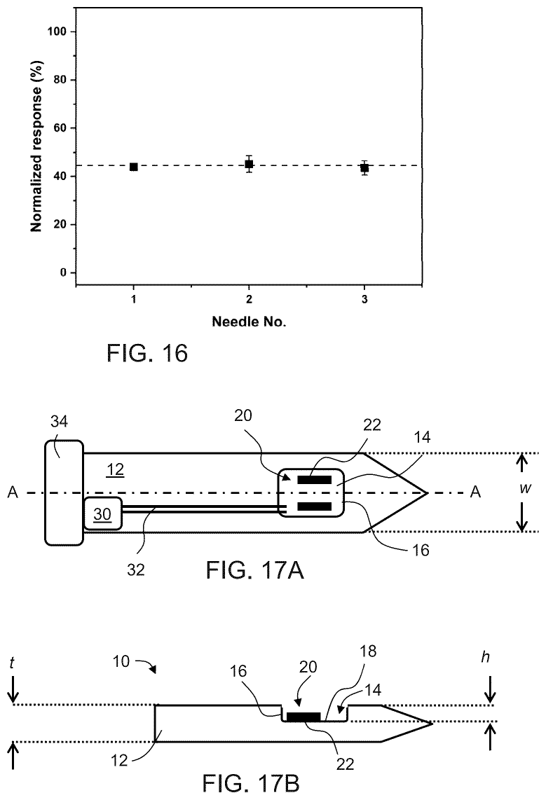

The response from the sensing microneedle array is shown to be highly accurate and reliable, FIG. 5H, making it useful as a POC diagnostic tool. FIG. 16, which is derived from FIG. 5H, shows the variation between each needle. Measurements of PSA-spiked serum and buffer by the same device produced near-identical curves. Therefore, the microneedles arrays of the present embodiments are highly reliable, and produce extremely reproducible protein response. The difference between curves of different dies is expected to diminish with mass-production and process scale-up.

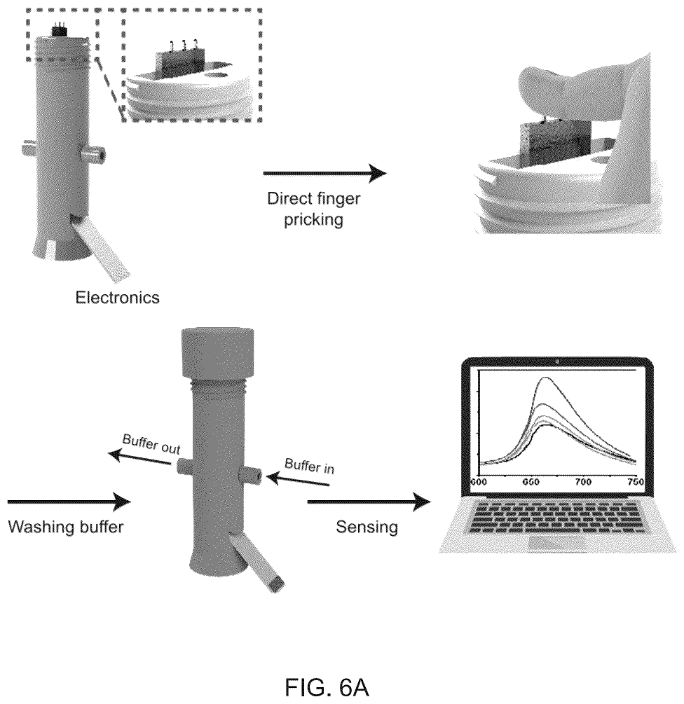

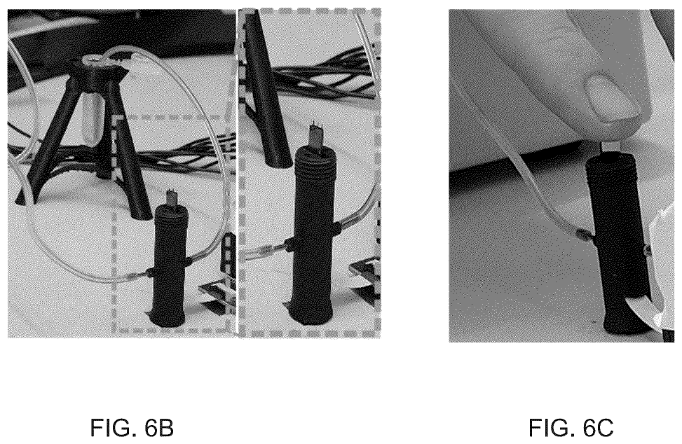

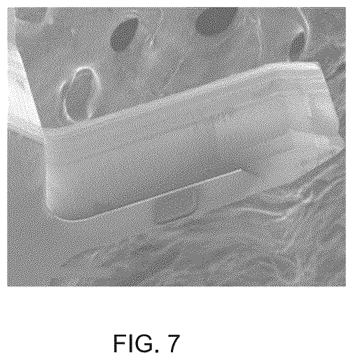

FIGS. 6A-C depict a laboratory-scale 3D printed prototype for the real-world application of the microneedle-based sensing platform for the finger-prick blood extraction-free intradermal protein biomarkers detection. The 3D-printed device allows for the subject to safely and comfortably prick his own finger, followed by the performance of the rapid and straightforward ex-dermal dissociation-based detection and quantification of the protein biomarkers of interest.

The presented blood extraction-free microneedle sensing platform provides an enormous conceptual leap in the field of medical diagnosis in general and POC medical diagnosis in particular. These fields are currently dominated by time-consuming invasive and extensive high-volume blood extraction and manipulation steps, performed mainly by professional medical staff at centralized facilities. The application of the microneedle-embedded chemically-modified nanostructure arrays of the present embodiments allows the simultaneous intradermal penetration and in-skin capillary blood-based biomarkers quantitative sampling and detection. This ultimate POC platform combines prominently advantageous attributes such as minimally-invasiveness, blood sample extraction-free, and samples manipulation-free requirements, clinically relevant high sensitivity and specificity, sensing accuracy, rapid detection turnover of under three minutes, multiplexing capabilities for the detection of multiple protein biomarkers based on a single-prick single-chip direct approach.

### CONCLUSIONS

This Example described a microneedle-embedded nanostructure array for the intradermal, minimally-invasive, and blood extraction-free platform for the clinical POC multiplexed detection of proteins biomarkers. The microneedle device of the present embodiments requires no extraction and ex-body manipulation procedures of large-volume venous blood samples, which is currently ordinary in all diagnostic tests.

The microneedle device of the present embodiments allows the direct intradermal probing of the prick-triggered capillary blood pool formed by the microneedle in the puncture site, preferably only a few hundreds of nanoliters in volume, and the concomitant in-skin quantitative capturing of the protein biomarkers of interest, followed by the microneedle removal and ex-vivo biomarkers levels quantification.

The microneedle of the present embodiments is preferably at least 1 mm length, and was shown to provide a nearly 100% success rate of reaching blood capillaries after each insertion event.

The microneedle device of the present embodiments has a micrometers-high SU-8-based open window structure which is useful for the physical protection of the molecular recognition layer. This protecting window allows skin penetration without the wiping off effect of the antibody recognition layer on the active sensing area. Still, this open window lets the fast and complete wetting of the sensing area when surrounded by the pricking-triggered capillary blood pool, thus allowing the free in-skin interaction of the embedded nanodevices sensing array with the surrounding blood sample.

The microneedles array of the present embodiments has shown a detection sensitivity in the sub-pM range and has been preliminary applied clinically for the intradermal direct in-vivo blood extraction-free detection of PSA on healthy human volunteers. These detection results directly correlate with values measured from venous blood extracted samples by gold-standard ELISA analysis.

The top-down process presented in this Example allows multiple vital advantages, such as high device redundancy for reliable results, ease of integration with future drug delivery applications, scalable and cost-effective process, and multiplex detection of multiple biomarkers in a single-prick single-chip device.

The microneedle device of the present embodiments can eliminate the need for currently well-established venous blood extraction and sample manipulation-based clinical approaches for disease diagnosis.

### Example 2

This Example provides additional experimental details for the experiments described in Example 1.

### Materials and Chemicals

8-inch SOI wafer (Silicon Valley Microelectronics), Acetone (9005-68, J. T. Baker), Isopropanol (9079-05, J. T. Baker), Deionized water (18 MΩ·cm), Phosphate buffer (PB, 10 mM, pH 8.5), Phosphate buffer (SB, 155 M, ˜pH 8.0), Phosphate buffer saline (PBS, 10 mM, pH 7.4, with 2.7 mM KCl and 137 mM NaCl), Glutaraldehyde solution (50 wt. % in H₂O, G7651, Sigma-Aldrich), (3-aminopropyl)-dimethyl-ethoxysilane (APDMES, SIA0603.0-5g, Gelest), Human PSA protein (ABCAM, ab78528) PSA antibody (ABCAM, ab75684), Cardiac Troponin I protein (cTnI, ABCAM, ab207624), Cardiac Troponin I antibody (ABCAM, ab38210), GFP Protein (ABCAM, ab84191), GFP antibody (ABCAM, ab1218) Alexa-488 NHS (A20000, Thermo Fisher), PDMS (Sylgard), LOR5A (Kayaku Advanced Materials), LOR7A (Kayaku Advanced Materials), LOR10A (Kayaku Advanced Materials), SU8 2000.5 (Kayaku Advanced Materials), SU8 3005 (Kayaku Advanced Materials), AZ1505 (MicroChemicals), AZ4562 (MicroChemicals), PMGI SF15 (Kayaku Advanced Materials), Buffered Oxide Etchant 6:1 (BOE, Transene), Gold Etchant TFE (Transene), Chromium Cermet Etchant (Transene), N-methyl-2-pyrrolidone (NMP, J. T. Baker), Hydrogen peroxide (30% in water, Bio-Lab), Sulfuric acid (95-98%, Bio-Lab), Methylmethacrylate (MMA, EL6, Kayaku Advanced Materials), Polymethylmethacrylate (PMMA, A4, Kayaku Advanced Materials), AZ726 (MicroChemicals), Methyl isobutyl ketone (MIBK 1:3, Kayaku Advanced Materials), Hydrofluoric acid (48%, Sigma-Aldrich), Tetramethylammonium hydroxide (10% in water, Sigma-Aldrich), AZ400K (MicroChemicals).

### Nanowire Fabrication

30 mm×30 mm SOI dies were thoroughly cleaned using acetone, IPA, and DIW and were dipped in a 1:3 H₂O₂:H₂SO₄ piranha solution for 2 minutes. Following a 2 minute 60W O₂ plasma, the dies were dipped in a 6:1 BOE to remove the native oxide and were thoroughly washed with DIW. LOR5A and AZ1505 were spin-coated on the dies using 500 rpm for 5 seconds, followed by 4000 rpm for 45 seconds. The dies were baked at 180° C. for 5 minutes following the LOR5A spin coat and were baked at 100° C. for 1 minute following the AZ1505 spin coat process. E-beam markers were exposed using UV lithography and were developed for 1 minute in AZ726 (MicroChemicals), followed by a thorough wash in DIW. The markers were evaporated with 5 nm Cr and 30 nm Au and put in NMP to resist lift-off.

Once done, the dies were coated with MMA EL6 and PMMA A4, using 500 rpm for 3 seconds and 5000 rpm for 60 seconds. The die was baked at 180° C. for 3 minutes and 1 minute, respectively. E-beam lithography (Raith 150) with 10 kV was used to expose the PMMA layer, with the wires exposed using 10 μm aperture and 140 μC/cm² and the pads exposed using 60 μm aperture and 120 μC/cm². Development took place using 1:3 MIBK (methyl isobutyl ketone):IPA solution for 1 minute, followed by a thorough rinse in IPA. The exposed wire pattern was evaporated with 5 nm Cr and 30 nm Au and was placed in acetone for lift-off.

The dies were cleaned in IPA and DIW and were put in 60W 02 plasma for 2 minutes. The native oxide was removed using a 1:9 diluted 48% HF solution for 10 seconds and was directly placed, without rinsing, in a 10% TMAH solution heated to 75° C. After approximately 30 seconds, the die changed its color, indicating the dissolution of the device layer, and was rinsed in DIW. The Au and Cr were removed using appropriate etchants for 2 minutes each while thoroughly washing in DIW in between etchants.

### Electrodes Fabrication

The dies were spin-coated with LOR5A and AZ1505 as described above. Outer pads were exposed and developed in AZ726 for 1 minute, followed by a thorough rinse in DIW. The outer pads were evaporated with 5 nm Cr and 60 nm Au and were placed in warm NMP for lift-off. The dies were then washed in acetone, IPA, and DIW and were placed in an ozone generator for 3 minutes. LOR7A and AZ1505 were spin-coated as described above. Inner pads were exposed and developed as the outer pads. The inner pads were evaporated using 10 nm Ti, 90 nm Pd, and 5 nm Ti and were placed for the passivation process prior to lift off. The passivation took place via approximately 80 nm SiO₂ deposition using 200W ICP, 30W Bias, 95 mtorr, 80° C., 140 sccm N₂O, and 14 sccm 2% SiH₄/Ar for 20 minutes in a plasma-enhanced chemical vapor deposition (PECVD, Axis Benchmark 800 ICP) system. The dies were then placed in warm NMP for lift-off. Once done, rapid thermal processing (RTP, AnnealSys) was used to create ohmic contacts between the Ti/Si interface. The dies were heated to 450° C. in 5 seconds and remained for an extra 20 seconds in a forming gas environment (2% H₂ in N₂).

### Crevice Fabrication and Needles Thinning

SU8 was used. SU8 2000.5 was spin-coated using 300 rpm for 5 seconds, following 3000 rpm for 30 seconds. The die was baked for 5 minutes at 95° C. After UV exposure, the dies were baked for 1 minute at 95° C. The dies were developed in a designated SU8 developer for 1 minute, followed by an IPA rinse. Consecutively, SU8 3005 was coated using the same program as above. The die was baked for 1 minute at 65° C., following 10 minutes at 95° C. After exposure, the dies were baked for 4 minutes at 95° C. and developed as above.

LOR10A was dispensed to protect the nanowire region from the dicing operation, as the dicing is done on the backside. The dies were thinned using automatic dicer (Disco DAD 3350) via lowering the saw up to a depth that leaves approximately 250 μm thickness (around 500 μm) and moving laterally in steps smaller than the total width of the saw (e.g., if the saw was 200 μm thick, the steps were set to 90 μm). Once done, LOR10A was removed in NMP.

### Needles Formation

Prior to the deep silicon etching step, the BOX layer was removed. PMGI SF-15 was spin-coated at 500 rpm for 5 seconds and 1500 rpm for 45 seconds and was baked at 180° C. for 5 minutes. AZ4562 photoresist was spin-coated using the same parameters and baked at 115° C. for 1.5 minutes. The etch mask was exposed five consecutive times with 25 seconds stops in between. The dies were placed in DIW for 4 minutes following the exposure and developed in 1:2.5 diluted AZ400K developer for 4 minutes. The oxide layer was removed using reactive ion etching (RIE, Oerlikon) using 200W forward bias, 40 sccm CF₄, 5 sccm O₂, and 6 sccm Ar for 23 minutes at room temperature. The complete removal of the oxide was determined via an interferometer. The resists were removed in NMP.

To protect the fabricated SiNW from possible ion damage, the thick resist was applied before deep reactive ion etching (DRIE, Deep RIE Versaline DSE). PMGI SF-15 was spin-coated, as discussed above. Two layers of AZ4562 were spin-coated using the same spin parameters as above. The first layer was baked at 90° C. for 3 minutes, and the second layer was baked at 115° C. for 2.5 minutes. Once the etch mask was exposed, as discussed above, the dies were placed in DIW for 5 minutes and were developed in 1:2.5 diluted AZ400K developer for 8 minutes. After development, the dies were subjected to flood exposure of 400 mJ/cm². The dies were placed in the DRIE using heatsink grease (Dow Corning 340 Heat Sink Compound Grease) and were etched using a 3-step process for 300 loops. Once done, the dies were diced and separated into individual microneedle array sensors, and the remaining resists were removed in warm NMP.

### Antibody Modification

Prior to the modification process, the microneedle array sensor was mounted on a 3D printed holder (Form3 printer, Fromlabs), and was wire bonded to a flexible PCB. The mounted sensor was then placed in the ozone generator for 7 minutes to generate silanol groups on the SiNWs surface. The mounted array was placed in 200 μl of 95% APDMES solution for 1 hour in an Ar-filled glovebox. The sensor was then placed in 150 μl toluene to wash the remaining APDMES solution and was thoroughly rinsed with IPA and placed at 75° C. for 30 minutes to evaporate the remaining solvents completely.

Phosphate buffer (PB) was prepared by mixing 10 mM potassium phosphate monobasic solution and 10 mM potassium phosphate dibasic solution to pH 8.5. 1 ml of 50% Glutaraldehyde solution (Sigma Aldrich) was diluted in 5 ml of prepared PB with 50 mg of added NaCNBH₃. The microneedle array sensor was dipped in 200 μl of the above solution for 1 hour and was consecutively rinsed with DIW, IPA, and DIW again.

Anti-human PSA in 0.030 mg/ml concentration was used for the modification. The antibody was first centrifuged in a desalting column to clean and purify the protein properly. The anti-PSA was diluted to 30 μg/ml for the modification using a prepared solution of 5 ml PB and 50 mg of NaCNBH₃. The microneedle array was dipped in 200 μl of the antibody solution and was placed at 4° C. overnight.

Blocking solution was prepared using 150 μl of ethanolamine in 20 ml PB with 50 mg NaCNBH₃, which was titrated back to pH 8.5 using HCl. 200 μl of the above solution was used for 2 hours to block all unreacted aldehyde groups. The microneedle array was then thoroughly washed in PB by placing the sensor in 200 μL solution of clean PB for 10 minutes. This process was repeated a total of three times before sensing experiments.

Anti-GFP and anti-cTnI were modified using the same method and the same concentrations to verify the viability of the modification properly.

### Array Cleaning Prior to Skin Insertion

The microneedle arrays were washed well in autoclaved PB. The insertion area was sterilized by rubbing ethanol.

### In-Vitro and In-Vivo Electrical Measurements

Electrical measurements were performed by varying the gate voltage in order to select a gate voltage that provides a detectable change in current as a factor of concentration changes. The gate voltage was varied between −0.7V-0.3V, and the source-drain voltage was constantly applied using 0.2V. The in vitro measurements took place either in 1× phosphate buffer saline (PBS) or as received bovine serum. The microneedle array sensor was placed inside an Eppendorf containing 2 ml solution of either unspiked (‘clean’) or PSA-spiked solutions in different concentrations until stabilization (approximately 8 minutes). The desorption took place in a 5% EG solution in sensing buffer (phosphate buffer diluted by a factor of 100) using 2 ml solutions as well, until stabilization (approximately 10 minutes). In vivo measurements in capillary blood were performed by full penetration of the microneedle array into the volunteer's skin (arm or fingertip), with the microneedle probing allowed to occur for 1 minute before final microneedle removal, followed by the final quantitative sensing analysis.

### Material Characterization

XPS measurements were performed using the 5600 Multi-Technique System (PHI, USA). SEM images were taken using Environmental SEM (Quanta 200FEG, Jeol Co.).

### ELISA Measurements

ELISA kit to quantify total PSA was purchased from ABCAM (ab113327). The measurement protocol is as follows:

A 96-well plate coated with an antibody specific for Human PSA was used. 100 μl of standard solutions and samples (see elaborated below) were pipetted into the wells. The wells were washed thoroughly, and a 100 μl of biotinylated secondary antibody to Human PSA was added. The wells were washed thoroughly again; then 100 μl of HRP-conjugated streptavidin was added to each well. The wells were again washed, and 100 μl of TMB substrate solution was added, developing a blue color in proportion to the amount of PSA bound. 50 μl of Stop Solution changes the color from blue to yellow, and the intensity of the color is measured at 450 nm.

Approximately 5 ml of venous blood was extracted and centrifuged to coagulate and remove the red blood cells. The test was performed directly on the separated plasma fluid remaining after 2-fold dilution in Assay Diluent B provided in the kit.

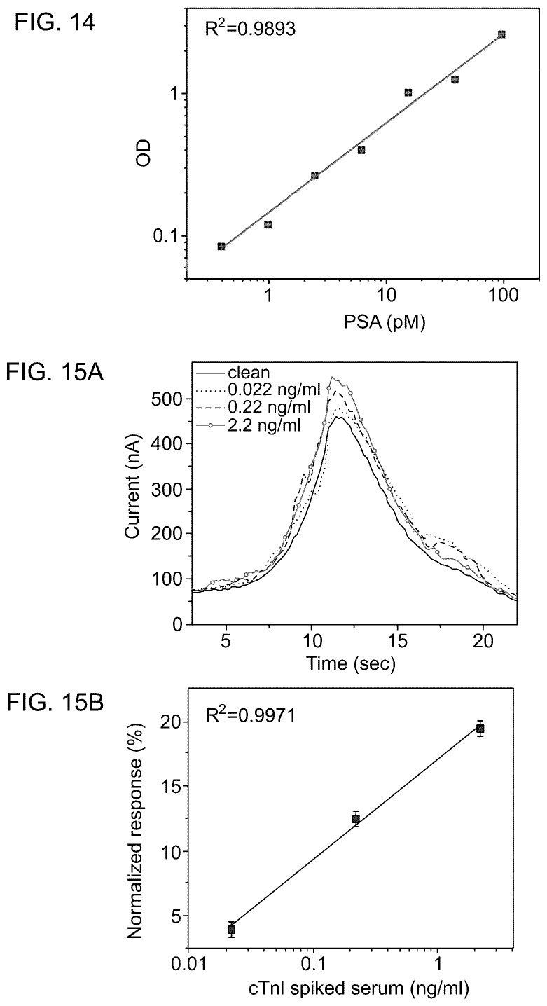

Standard PSA solution of 50,000 μg/ml was diluted in Assay Diluent B to perform calibration curve measurements of 10.24-2500 μg/ml (see FIG. 14).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] Yu, H.; Lu, Y.; Zhou, Y.; Wang, F.; He, F.; Xia, X. A Simple, Disposable Microfluidic Device for Rapid Protein Concentration and Purification via Direct-Printing. Lab Chip 2008, 8, 1496-1501.
[2] Kim, P.; Kim, S. J.; Han, J.; Suh, K. Y. Stabilization of Ion Concentration Polarization Using a Heterogeneous Nanoporous Junction. Nano Lett. 2009, 10, 16-23.
[3] Dhopeshwarkar, R.; Crooks, R. M.; Dzmitry Hlushkou, A.; Ulrich Tallarek. Transient Effects on Microchannel Electrokinetic Filtering with an Ion-Permselective Membrane. Anal. Chem. 2008, 80, 1039-1048.
[4] Jeun, M.; Lee, H. J.; Park, S.; Do, E.; Choi, J.; Sung, Y.-N.; Hong, S.-M.; Kim, S.-Y.; Kim, D.-H.; Kang, J. Y.; Son, H.-N.; Joo, J.; Song, E. M.; Hwang, S. W.; Park, S. H.; Yang, D.-H.; Ye, B. D.; Byeon, J.-S.; Choe, J.; Yang, S.-K. et al. A Novel Blood-Based Colorectal Cancer Diagnostic Technology Using Electrical Detection of Colon Cancer Secreted Protein-2. Adv. Sci. 2019, 6, 1802115.
[5] Gao, X.; Boryczka, J.; Kasani, S.; Wu, N. Enabling Direct Protein Detection in a Drop of Whole Blood with an “On-Strip” Plasma Separation Unit in a Paper-Based Lateral Flow Strip. Anal. Chem. 2020, 93, 1326-1332.
[6] Biagini, R. E.; Sammons, D. L.; Smith, J. P.; MacKenzie, B. A.; Striley, C. A. F.; Snawder, J. E.; Robertson, S. A.; Quinn, C. P. Rapid, Sensitive, and Specific Lateral-Flow Immunochromatographic Device to Measure Anti-Anthrax Protective Antigen Immunoglobulin G in Serum and Whole Blood. Clin. Vaccine Immunol. 2006, 13, 541-546.
[7] Espy, R. D.; Manicke, N. E.; Ouyang, Z.; Cooks, R. G. Rapid Analysis of Whole Blood by Paper Spray Mass Spectrometry for Point-of-Care Therapeutic Drug Monitoring. Analyst 2012, 137, 2344-2349.
[8] McCaughey, E. J.; Vecellio, E.; Lake, R.; Li, L.; Burnett, L.; Chesher, D.; Braye, S.; Mackay, M.; Gay, S.; Badrick, T. C.; Westbrook, J. I.; Georgiou, A. Current Methods of Haemolysis Detection and Reporting as a Source of Risk to Patient Safety: A Narrative Review. Clin. Biochem. Rev. 2016, 37, 143.
[9] Heireman, L.; Van Geel, P.; Musger, L.; Heylen, E.; Uyttenbroeck, W.; Mahieu, B. Causes, Consequences and Management of Sample Hemolysis in the Clinical Laboratory. Clin. Biochem. 2017, 50, 1317-1322.
[10] Wu, C. C.; Lin, H. Y.; Wang, C. P.; Lu, L. F.; Yu, T. H.; Hung, W. C.; Houng, J. Y.; Chung, F. M.; Lee, Y. J.; Hu, J. J. Evaluation of a Rapid Quantitative Determination Method of PSA Concentration with Gold Immunochromatographic Strips. BMC Urol. 2015, 15, 109.
[11] Kemper, D. W.; Semjonow, V.; de Theije, F.; Keizer, D.; van Lippen, L.; Mair, J.; Wille, B.; Christ, M.; Geier, F.; Hausfater, P.; Pariente, D.; Scharnhorst, V.; Curvers, J.; Nieuwenhuis, J. Analytical Evaluation of a New Point of Care System for Measuring Cardiac Troponin I. Clin. Biochem. 2017, 50, 174-180.
[12] Lippi, G.; Plebani, M.; Di Somma, S.; Cervellin, G. Hemolyzed Specimens: A Major Challenge for Emergency Departments and Clinical Laboratories. Crit. Rev. Clin. Lab. Sci. 2011, 48, 143-153.
[13] Sawant, R. B.; Jathar, S. K.; Rajadhyaksha, S. B.; Kadam, P. T. Red Cell Hemolysis during Processing and Storage. Asian J. Transfus. Sci. 2007, 1, 47.
[14] Sharma, S.; Huang, Z.; Rogers, M.; Boutelle, M.; Cass, A. E. G. Evaluation of a Minimally Invasive Glucose Biosensor for Continuous Tissue Monitoring. Anal. Bioanal. Chem. 2016 40829 2016, 408, 8427-8435.
[15] Bal, S. M.; Caussin, J.; Pavel, S.; Bouwstra, J. A. In Vivo Assessment of Safety of Microneedle Arrays in Human Skin. Eur. J. Pharm. Sci. 2008, 35 (3), 193-202.
[16] Noh, Y. W.; Kim, T. H.; Baek, J. S.; Park, H. H.; Lee, S. S.; Han, M.; Shin, S. C.; Cho, C. W. In Vitro Characterization of the Invasiveness of Polymer Microneedle against Skin. Int. J. Pharm. 2010, 397, 201-205.
[17] El-Laboudi, A.; Oliver, N. S.; Cass, A.; Johnston, D. Use of Microneedle Array Devices for Continuous Glucose Monitoring: A Review. Diabetes Technol. Ther. 2013, 15, 101-115.
[18] Yu, J.; Wang, J.; Zhang, Y.; Chen, G.; Mao, W.; Ye, Y.; Kahkoska, A. R.; Buse, J. B.; Langer, R.; Gu, Z. Glucose-Responsive Insulin Patch for the Regulation of Blood Glucose in Mice and Minipigs. Nat. Biomed. Eng. 2020, 4, 499-506.
[19] Yu, J.; Zhang, Y.; Ye, Y.; DiSanto, R.; Sun, W.; Ranson, D.; Ligler, F. S.; Buse, J. B.; Gu, Z. Microneedle-Array Patches Loaded with Hypoxia-Sensitive Vesicles Provide Fast Glucose-Responsive Insulin Delivery. Proc. Natl. Acad. Sci. 2015, 112, 8260-8265.
[20] Yoon, Y.; Lee, G. S.; Yoo, K.; Lee, J.-B. Fabrication of a Microneedle/CNT Hierarchical Micro/Nano Surface Electrochemical Sensor and Its In-Vitro Glucose Sensing Characterization. Sensors 2013, 13, 16672-16681.
[21] Wang, P. M.; Cornwell, M.; Prausnitz, M. R. Minimally Invasive Extraction of Dermal Interstitial Fluid for Glucose Monitoring Using Microneedles. DIABETES Technol. Ther. 2005, 7.
[22] Miller, P. R.; Taylor, R. M.; Tran, B. Q.; Boyd, G.; Glaros, T.; Chavez, V. H.; Krishnakumar, R.; Sinha, A.; Poorey, K.; Williams, K. P.; Branda, S. S.; Baca, J. T.; Polsky, R. Extraction and Biomolecular Analysis of Dermal Interstitial Fluid Collected with Hollow Microneedles. Commun. Biol. 2018 11 2018, 1, 1-11.
[23] Teymourian, H.; Tehrani, F.; Mahato, K.; Wang, J. Lab under the Skin: Microneedle Based Wearable Devices. Adv. Healthc. Mater. 2021, 10, 2002255.
[24] Heifler, O.; Borberg, E.; Harpak, N.; Zverzhinetsky, M.; Krivitsky, V.; Gabriel, I.; Fourman, V.; Sherman, D.; Patolsky, F. Clinic-on-a-Needle Array toward Future Minimally Invasive Wearable Artificial Pancreas Applications. ACS Nano 2021, 15, 12019-12033.
[25] Mishra, R. K.; Mohan, A. M. V.; Soto, F.; Chrostowski, R.; Wang, J. A Microneedle Biosensor for Minimally-Invasive Transdermal Detection of Nerve Agents. Analyst 2017, 142, 918-924.
[26] Corrie, S. R.; Fernando, G. J. P.; Crichton, M. L.; Brunck, M. E. G.; Anderson, C. D.; Kendall, M. A. F. Surface-Modified Microprojection Arrays for Intradermal Biomarker Capture, with Low Non-Specific Protein Binding. Lab Chip 2010, 10, 2655-2658.
[27] Sharma, S.; El-Laboudi, A.; Reddy, M.; Jugnee, N.; Sivasubramaniyam, S.; Sharkawy, M. El; Georgiou, P.; Johnston, D.; Oliver, N.; Cass, A. E. G. A Pilot Study in Humans of Microneedle Sensor Arrays for Continuous Glucose Monitoring. Anal. Methods 2018, 10, 2088-2095.
[28] Romanyuk, A. V.; Zvezdin, V. N.; Samant, P.; Grenader, M. I.; Zemlyanova, M.; Prausnitz, M. R. Collection of Analytes from Microneedle Patches. Anal. Chem. 2014, 86, 10520-10523.
[29] Dinesh, B.; Saraswathi, R. Electrochemical Synthesis of Nanostructured Copper-Curcumin Complex and Its Electrocatalytic Application towards Reduction of 4-Nitrophenol. Sensors Actuators B Chem. 2017, 253, 502-512.
[30] Ishai, M. Ben; Patolsky, F. Shape-and Dimension-Controlled Single-Crystalline Silicon and Sige Nanotubes: Toward Nanofluidic Fet Devices. J. Am. Chem. Soc. 2009, 131, 3679-3689.
[31] Ben-Ishai, M.; Patolsky, F. A Route to High-Quality Crystalline Coaxial Core/Multishell Ge@Si(GeSi)n and Si@(GeSi)n Nanowire Heterostructures. Adv. Mater. 2010, 22, 902-906.
[32] Pevzner, A.; Engel, Y.; Elnathan, R.; Tsukernik, A.; Barkay, Z.; Patolsky, F. Confinement-Guided Shaping of Semiconductor Nanowires and Nanoribbons: “Writing with Nanowires.” Nano Lett. 2012, 12, 7-12.
[33] Weizmann, Y.; Patolsky, F.; Popov, I.; Willner, I. Telomerase-Generated Templates for the Growing of Metal Nanowires. Nano Lett. 2004, 4, 787-792.
[34] Jiang, X.; Xiong, Q.; Nam, S.; Qian, F.; Li, Y.; Lieber, C. M. InAs/InP Radial Nanowire Heterostructures as High Electron Mobility Devices. Nano Lett. 2007, 7, 3214-3218.
[35] Harpak, N.; Davidi, G.; Granot, E.; Patolsky, F. Diversely Doped Uniform Silicon Nanotube Axial Heterostructures Enabled by “Dopant Reflection.” Langmuir 2021, 37, 1247-1254.
[36] Kuhn, S.; Asenbaum, P.; Kosloff, A.; Sclafani, M.; Stickler, B. A.; Nimmrichter, S.; Hornberger, K.; Cheshnovsky, O.; Patolsky, F.; Arndt, M. Cavity-Assisted Manipulation of Freely Rotating Silicon Nanorods in High Vacuum. Nano Lett. 2015, 15, 5604-5608.
[37] Yeor-Davidi, E.; Zverzhinetsky, M.; Krivitsky, V.; Patolsky, F. Real-Time Monitoring of Bacterial Biofilms Metabolic Activity by a Redox-Reactive Nanosensors Array. J. Nanobiotechnology 2020, 18, 1-11.
[38] Borberg, E.; Pashko, S.; Koren, V.; Burstein, L.; Patolsky, F. Depletion of Highly Abundant Protein Species from Biosamples by the Use of a Branched Silicon Nanopillar On-Chip Platform. Anal. Chem. 2021, 93, 14527-14536.
[39] Harpak, N.; Davidi, G.; Schneier, D.; Menkin, S.; Mados, E.; Golodnitsky, D.; Peled, E.; Patolsky, F. Large-Scale Self-Catalyzed Spongelike Silicon Nano-Network-Based 3D Anodes for High-Capacity Lithium-Ion Batteries. Nano Lett. 2019, 19, 1944-1954.
[40] Harpak, N.; Davidi, G.; Melamed, Y.; Cohen, A.; Patolsky, F. Self-Catalyzed Vertically Aligned Carbon Nanotube-Silicon Core-Shell Array for Highly Stable, High-Capacity Lithium-Ion Batteries. Langmuir 2020, 36, 889-896.
[41] Harpak, N.; Davidi, G.; Cohen, A.; Raz, A.; Patolsky, F. Thermally-Treated Nanowire-Structured Stainless-Steel as an Attractive Cathode Material for Lithium-Ion Batteries. Nano Energy 2020, 76, 105054.
[42] Patolsky, F.; Zheng, G.; Hayden, O.; Lakadamyali, M.; Zhuang, X.; Lieber, C. M. Electrical Detection of Single Viruses. Proc. Natl. Acad. Sci. 2004, 101, 14017-14022.
[43] Zheng, G.; Patolsky, F.; Cui, Y.; Wang, W. U.; Lieber, C. M. Multiplexed Electrical Detection of Cancer Markers with Nanowire Sensor Arrays. Nat. Biotechnol. 2005 2310 2005, 23, 1294-1301.
[44] Krivitsky, V.; Zverzhinetsky, M.; Krivitsky, A.; Hsiung, L.-C.; Naddaka, V.; Gabriel, I.; Lefler, S.; Conroy, J.; Burstein, L.; Patolsky, F. Cellular Metabolomics by a Universal Redox-Reactive Nanosensors Array: From the Cell Level to Tumor-on-a-Chip Analysis. Nano Lett. 2019, 19, 2478-2488.
[45] Lichtenstein, A.; Havivi, E.; Shacham, R.; Hahamy, E.; Leibovich, R.; Pevzner, A.; Krivitsky, V.; Davivi, G.; Presman, I.; Elnathan, R.; Engel, Y.; Flaxer, E.; Patolsky, F. Supersensitive Fingerprinting of Explosives by Chemically Modified Nanosensors Arrays. Nat. Commun. 2014, 5, 1-12.
[46] Meir, R.; Zverzhinetsky, M.; Harpak, N.; Borberg, E.; Burstein, L.; Zeiri, O.; Krivitsky, V.; Patolsky, F. Direct Detection of Uranyl in Urine by Dissociation from Aptamer-Modified Nanosensor Arrays. Anal. Chem. 2020, 92, 12528-12537.
[47] Zverzhinetsky, M.; Krivitsky, V.; Patolsky, F. Direct Whole Blood Analysis by the Antigen-Antibody Chemically-Delayed Dissociation from Nanosensors Arrays. Biosens. Bioelectron. 2020, 170, 112658.
[48] Krivitsky, V.; Zverzhinetsky, M.; Patolsky, F. Antigen-Dissociation from Antibody-Modified Nanotransistor Sensor Arrays as a Direct Biomarker Detection Method in Unprocessed Biosamples. Nano Lett. 2016, 16, 6272-6281.
[49] Borberg, E.; Zverzhinetsky, M.; Krivitsky, A.; Kosloff, A.; Heifler, O.; Degabli, G.; Soroka, H. P.; Fainaro, R. S.; Burstein, L.; Reuveni, S.; Diamant, H.; Krivitsky, V.; Patolsky, F. Light-Controlled Selective Collection-and-Release of Biomolecules by an On-Chip Nanostructured Device. Nano Lett. 2019, 19, 5868-5878.
[50] Krivitsky, V.; Hsiung, L. C.; Lichtenstein, A.; Brudnik, B.; Kantaev, R.; Elnathan, R.; Pevzner, A.; Khatchtourints, A.; Patolsky, F. Si Nanowires Forest-Based on-Chip Biomolecular Filtering, Separation and Preconcentration Devices: Nanowires Do It All. Nano Lett. 2012, 12, 4748-4756.
[51] Yang, X.; Gao, A.; Wang, Y.; Li, T. Wafer-Level and Highly Controllable Fabricated Silicon Nanowire Transistor Arrays on (111) Silicon-on-Insulator (SOI) Wafers for Highly Sensitive Detection in Liquid and Gaseous Environments. Nano Res. 2018 113 2018, 11, 1520-1529.
[52] Li, D.; Chen, H.; Fan, K.; Labunov, V.; Lazarouk, S.; Yue, X.; Liu, C.; Yang, X.; Dong, L.; Wang, G. A Supersensitive Silicon Nanowire Array Biosensor for Quantitating Tumor Marker CtDNA. Biosens. Bioelectron. 2021, 181, 113147.
[53]A. Malsagova, K.; D. Ivanov, Y.; O. Pleshakova, T.; L. Kaysheva, A.; D. Shumov, I.; F. Kozlov, A.; I. Archakov, A.; P. Popov, V.; I. Fomin, B.; V. Latyshev, A. A SOI-Nanowire Biosensor for the Multiple Detection of D-NFATc1 Protein in the Serum. Anal. Methods 2015, 7, 8078-8085.
[54] Stem, E.; Vacic, A.; Reed, M. A. Semiconducting Nanowire Field-Effect Transistor Biomolecular Sensors. IEEE Trans. Electron Devices 2008, 55, 3119-3130.
[55] Beckman, R. A.; Johnston-Halperin, E.; Melosh, N. A.; Luo, Y.; Green, J. E.; Heath, J. R. Fabrication of Conducting Si Nanowire Arrays. J. Appl. Phys. 2004, 96, 5921.
[56] Palsdottir, T.; Nordstrom, T.; Karlsson, A.; Granberg, H.; Clements, M.; Eklund, M. The Impact of Different Prostate-Specific Antigen (PSA) Testing Intervals on Gleason Score at Diagnosis and the Risk of Experiencing False-Positive Biopsy Recommendations: A Population-Based Cohort Study. BMJ Open 2019, 9.
[57] Esteban, E. P.; Almodovar-Abreu, L. A New Interpretation of the Standard PSA-Test. Res. Reports Urol. 2020, 12, 75-84.
[58] Zhang, D.; Lu, Y.; Zhang, Q.; Liu, L.; Li, S.; Yao, Y.; Jiang, J.; Liu, G. L.; Liu, Q. Protein Detecting with Smartphone-Controlled Electrochemical Impedance Spectroscopy for Point-of-Care Applications. Sensors Actuators B Chem. 2016, 222, 994-1002.
[59] Goh, C. M.; Meriaudeau, F.; Saad, N. M.; Subramaniam, R.; Ali, S. A. Subcutaneous Veins Depth Measurement Using Diffuse Reflectance Images. Opt. Express 2017, 25, 25741-25759.
[60] Oltulu, P.; Ince, B.; Kökbudak, N.; Findik, S.; Kiliç, F. Measurement of Epidermis, Dermis, and Total Skin Thicknesses from Six Different Body Regions with a New Ethical Histometric Technique. Turkish J. Plast. Surg. 2018, 26, 56.
[61] Gill, H. S.; Denson, D. D.; Burris, B. A.; Prausnitz, M. R. Effect of Microneedle Design on Pain in Human Subjects. Clin. J. Pain 2008, 24, 585.
[62] Mohammed, B. S.; Cameron, G. A.; Cameron, L.; Hawksworth, G. H.; Helms, P. J.; McLay, J. S. Can Finger-Prick Sampling Replace Venous Sampling to Determine the Pharmacokinetic Profile of Oral Paracetamol?Br. J. Clin. Pharmacol. 2010, 70, 52-56.
[63] Azzouzi, A.-R.; Larre, S.; Cormier, L.; Roupret, M.; Valeri, A.; Mangin, P.; Berthon, P.; Villette, J.-M.; Fiet, J.; Cussenot, O. Relevance of the Prostate-Specific Antigen (PSA) Nanotest Compared to the Classical PSA Test in the Organized Mass Screening of Prostate Cancer. BJU Int. 2007, 99, 762-764.
[64] Miano, R.; Mele, G. O.; Germani, S.; Bove, P.; Sansalone, S.; Pugliese, P. F.; Micali, F. Evaluation of a New, Rapid, Qualitative, One-Step PSA Test for Prostate Cancer Screening: The PSA RapidScreen Test. Prostate Cancer Prostatic Dis. 2005 83 2005, 8, 219-223.
[65] Aellen, J.; Dabiri, A.; Heim, A.; Liaudet, L.; Burnier, M.; Ruiz, J.; Feihl, F.; Waeber, B. Preserved Capillary Density of Dorsal Finger Skin in Treated Hypertensive Patients with or without Type 2 Diabetes. Microcirculation 2012, 19, 554-562.
[66] Debbabi, H.; Uzan, L.; Mourad, J. J.; Safar, M.; Levy, B. I.; Tibiriga, E. Increased Skin Capillary Density in Treated Essential Hypertensive Patients. Am. J. Hypertens. 2006, 19, 477-483.
[67] Houben, A. J. H. M.; Martens, R. J. H.; Stehouwer, C. D. A. Assessing Microvascular Function in Humans from a Chronic Disease Perspective. J. Am. Soc. Nephrol. 2017, 28, 3461-3472.
[68] Monticone, G.; Colonna, L.; Palermi, G.; Bono, R.; Puddu, P. Quantitative Nailfold Capillary Microscopy Findings in Patients with Acrocyanosis Compared with Patients Having Systemic Sclerosis and Control Subjects. J. Am. Acad. Dermatol. 2000, 42, 787-790.
[69] Tibiriçá, E.; Rodrigues, E.; Cobas, R.; Gomes, M. B. Increased Functional and Structural Skin Capillary Density in Type 1 Diabetes Patients with Vascular Complications. Diabetol. Metab. Syndr. 2009 11 2009, 1, 1-4.
[70] Jiang, S.; Li, P.; Yu, Y.; Liu, J.; Yang, Z. Experimental Study of Needle-Tissue Interaction Forces: Effect of Needle Geometries, Insertion Methods and Tissue Characteristics. J. Biomech. 2014, 47, 3344-3353.
[71] Ranamukhaarachchi, S. A.; Stoeber, B. Determining the Factors Affecting Dynamic Insertion of Microneedles into Skin. Biomed. Microdevices 2019, 21, 1-8.
[72] Peled, A.; Pevzner, A.; Peretz Soroka, H.; Patolsky, F. Morphological and Chemical Stability of Silicon Nanostructures and Their Molecular Overlayers under Physiological Conditions: Towards Long-Term Implantable Nanoelectronic Biosensors. J. Nanobiotechnology 2014 121 2014, 12, 1-11.
[73] Mejak, S. L.; Bayliss, J.; Hanks, S. D. Long Distance Bicycle Riding Causes Prostate-Specific Antigen to Increase in Men Aged 50 Years and Over. PLoS One 2013, 8, 56030.
[74] Jiandani, D.; Randhawa, A.; Brown, R. E.; Hamilton, R.; Matthew, A. G.; Kuk, J. L.; Alibhai, S. M. H.; Tufts, E.; Santa Mina, D. The Effect of Bicycling on PSA Levels: A Systematic Review and Meta-Analysis. Prostate Cancer Prostatic Dis. 2015, 18, 208-212.
[75] Mashkoor, F. C.; Al-Asadi, J. N.; Al-Naama, L. M. Serum Level of Prostate-Specific Antigen (PSA) in Women with Breast Cancer. Cancer Epidemiol. 2013, 37, 613-618.

1. A microneedle device, comprising:
a device structure, insertable to a living body and being formed with an opened niche at least partially surrounded by walls of micrometric heights above a base of said niche for allowing said niche to be filled with a blood sample upon said insertion;
a biosensor configured to sense a bioanalyte in said blood sample and comprising a sensing element formed on said base and having a thickness less than said micrometric heights.

2. The device of claim 1, comprising a plurality of biosensors each configured to sense a different bioanalyte in said blood sample.

3. The device of claim 2, wherein said device structure is formed with a plurality of opened niches, and wherein sensing elements of at least two of said biosensors are on bases of different niches.

4. The device of claim 2, wherein sensing elements of at least two of said biosensors are on a base of the same niche.

5. The device of claim 1, comprising an inlet fluidic port at a section of said structure that remains outside said body after said insertion, and a fluidic channel extending from said inlet port to said niche, for establishing a flow of washing buffer into said niche in situ.

6. The device of claim 1, comprising an electrical communication port in electrical communication with said biosensor for transmitting signal from said biosensor to a location outside said body, while said biosensor is inside said body.

7. A device for monitoring at least presence of a bioanalyte, comprising a substrate having a skin contact surface, and a plurality of microneedle devices outwardly protruding from said skin contact surface, wherein at least one of said microneedle devices comprises the device of claim 1.

8. The device of claim 1, wherein said bioanalyte comprises at least one of a protein, an miRNA, a free-DNA, an exosome, a metabolite, an antibody, and a receptor.

9-14. (canceled)

15. The device of claim 1, wherein said biosensor comprises a source electrode and a drain electrode formed on said base or said walls, and wherein said sensing element comprises a nanostructure having a sub-micrometric thickness connecting between said electrodes and being modified by an immobilized affinity moiety selected to interact with said bioanalyte to effect a change in an electrical property of said nanostructure.

16. The device of claim 15, wherein said biosensor is a transistor and wherein said nanostructure is a channel of said transistor.

17. The device of claim 15, wherein said affinity moiety comprises an immunogenic moiety.

18. The device of claim 17, wherein said immunogenic moiety comprises at least one of (i) an antibody or a fragment thereof, and (ii) an antigen wherein said bioanalyte comprises an antibody to said antigen.

19. (canceled)

20. The device of claim 15, wherein said affinity moiety comprises a ligand and said bioanalyte comprises a receptor.

21. A method of detecting a bioanalyte in the blood of a subject, the method comprising:
contacting the device of claim 1 with the blood of the subject in vivo;
extracting the device from the body of the subject; and
obtaining a signal from the biosensor thereby detecting a bioanalyte in the blood.

22. A method of detecting a bioanalyte in the blood of a subject, the method comprising:
contacting the device of claim 15 with the blood of the subject in vivo;
extracting the device from the body of the subject;
washing said biosensor; and
detecting the bioanalyte based on a detectable signal received from said biosensor within a time-window beginning a predetermined time period after a beginning time of said washing.

23. A method of detecting a bioanalyte in the blood of a subject, the method comprising:
contacting the device according to claim 5 with the blood of the subject in vivo;
washing said biosensor via said fluidic channel;
detecting the bioanalyte based on a detectable signal received from said biosensor within a time-window beginning a predetermined time period after a beginning time of said washing.

24. The method according to claim 23, wherein said device comprises an electrical communication port in electrical communication with said biosensor for transmitting signal from said biosensor to a location outside said body, and wherein the method comprises receiving said signal via said electrical communication port while said biosensor is inside said body.

25. The method according to claim 23, comprising extracting the device from the body of the subject, following said washing, and receiving said signal after said extraction.

26. The method of claim 22, wherein said detecting comprises excluding from said signal any portion generated before said time-window.

27. The method of claim 22, wherein said beginning of said time-window is defined at a time point at which a rate of change of said signal, in absolute value, is below a predetermined threshold.
